SEQUENCE LISTING (50 sequences)

SEQ ID NO: 1 - DNA, 2210 bp, Streptomyces avermitilis
cgctagctactatcgagtcagtcgaattctatcggtacatggaggcagtgatgggcgtggcggccggtccgattcgtgttgtagttgcaaaaccggggctggatggtcatgaccgcggtgctaaggtaatcgcgcgtgcactgcgtgacgctggcatggaagtcatttatacggggctgcaccagacgccggagcaagtggtcgacactgctatccaagaggacgctgatgccattggcctgagcatcctgtctggggcacataatacactgtttgctcgcgtgctggagctgctgaaagaacgcgatgcagaagatattaaagttttcggcgggggcatcattccggaggccgacatcgcaccgctgaaggaaaaaggtgtcgctgagatttttaccccgggcgcgacgactacaagtatcgtggaatgggtacgtggtaacgttcgccaagccgtgtaaggccatcgacttttactaggacgtcctagtaaattcgatggtatggacgcggacgccattgaagagggccgtcgccgttggcaggcgcgttacgataaagctcgcaagcgcgacgcggactttacgactctgtcgggcgatccggtagatccggtttacggtccgcgcccgggtgatacttatgacggcttcgaacgtattgggtggccgggtgaatacccgtttactcgcggcctgtatgccactggctaccgcggtcgtacatggactatccgccaattcgcagggtttggcaatgccgaacagaccaacgaacgttataaaatgatcctggccaatgggggcggcggtctgagcgtggccttcgatatgccgacgctgatgggtcgtgatagtgatgacccgcgctccctgggtgaggtagggcactgcggcgtggcaattgattccgcagccgacatggaagtgctgttcaaagacatcccgctgggggatgtcaccacgtcgatgaccattagtggcccggccgtaccggtgttctgcatgtatctggtagctgcggaacgccaaggtgtcgatccggctgtactgaacgggacactgcagacggacatctttaaggaatatattgcccagaaagaatggctgttccaaccggagccgcatctgcgtctgatcggtgatctgatggaacactgcgcacgcgatattccggcgtataaaccgctgtcggtcagtggttaccacatccgtgaggctggcgccactgccgcgcaggaactggcttatactctggccgatggttttgggtacgttgaactgggcctgtcacgcggtctggacgtcgatgttttcgccccgggtctgagtttcttttttgacgcgcacgttgatttcttcgaagagatcgccaaatttcgtgccgcacgccgtatctgggctcgttggctgcgcgatgagtatggcgccaaaacggagaaggcacagtggctgcgcttccacacgcaaactgctggtgtatcgctgactgcacaacagccgtacaacaatgtagtgcgtactgccgttgaggcgctggcagccgttctgggtgggacaaatagcctgcacacgaacgccctggacgagacactggcgctgccgtcggagcaagccgcagagatcgctctgcgcactcaacaggtactgatggaggaaacaggggtcgcgaacgtggcagacccgctggggggctcgtggtatatcgagcaactgaccgaccgtatcgaagcggatgccgaaaagatttttgagcagatccgtgaacgtggtcgccgcgcttgcccggatggccaacacccgattgggccgattacttcagggattctgcgcggtatcgaggacgggtggtttacgggggaaattgcggagtccgcattccaatatcagcgttcgctggagaaaggtgataaacgtgtagtgggtgttaactgcctggaaggtagtgttacaggggacctggagatcctgcgtgttagtcatgaagtagaacgtgagcaagtccgcgaactggccggccgcaagggtcgtcgcgatgacgcgcgtgtacgcgctagtctggatgcaatgctggccgcggctcgtgatggctcaaatatgattgcaccgatgctggaggccgtccgcgcggaagcaaccctgggggaaatctgtggggtgctgcgtgacgagtggggcgtttacgttgagccgccgggtttttaagatatc

SEQ ID NO: 2 - DNA, 2759 bp, Clostridium acetobutylicum
ctctcccgggtataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagacgcggccgcagag

SEQ ID NO: 3 - DNA, 4272 bp, artificial sequence
  Plasmid
atgaccatgattacgccaagcgcgcaattaaccctcactaaagggaacaaaagctgggtaccgggccccccctcgaggtcgacggtatcgataagcttgatatccactgtggaattcgcccttaagggcgaattccacattggtcgctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattagtgtccttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 4 - DNA, 1202 bp, Clostridium acetobutylicum
gaattcggaggagtaaaacatgagagatgtagtaatagtaagtgctgtaagaactgcaataggagcatatggaaaaacattaaaggatgtacctgcaacagagttaggagctatagtaataaaggaagctgtaagaagagctaatataaatccaaatgagattaatgaagttatttttggaaatgtacttcaagctggattaggccaaaacccagcaagacaagcagcagtaaaagcaggattacctttagaaacacctgcgtttacaatcaataaggtttgtggttcaggtttaagatctataagtttagcagctcaaattataaaagctggagatgctgataccattgtagtaggtggtatggaaaatatgtctagatcaccatatttgattaacaatcagagatggggtcaaagaatgggagatagtgaattagttgatgaaatgataaaggatggtttgtgggatgcatttaatggatatcatatgggagtaactgcagaaaatattgcagaacaatggaatataacaagagaagagcaagatgaattttcacttatgtcacaacaaaaagctgaaaaagccattaaaaatggagaatttaaggatgaaatagttcctgtattaataaagactaaaaaaggtgaaatagtctttgatcaagatgaatttcctagattcggaaacactattgaagcattaagaaaacttaaacctattttcaaggaaaatggtactgttacagcaggtaatgcatccggattaaatgatggagctgcagcactagtaataatgagcgctgataaagctaacgctctcggaataaaaccacttgctaagattacttcttacggatcatatggggtagatccatcaataatgggatatggagctttttatgcaactaaagctgccttagataaaattaatttaaaacctgaagacttagatttaattgaagctaacgaggcatatgcttctcaaagtatagcagtaactagagatttaaatttagatatgagtaaagttaatgttaatggtggagctatagcacttggacatccaataggtgcatctggtgcacgtattttagtaacattactatacgctatgcaaaaaagagattcaaaaaaaggtcttgctactctatgtattggtggaggtcagggaacagctctcgtagttgaaagagactaagctt

SEQ ID NO: 5 - DNA, 4050 bp, artificial sequence
  Polynucelotide construct comprising crt, bcd, etfB and etfA from C. acetobutylicum and hdb from C. beijerinckii.
atcccgggatattttaggaggattagtcatggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagataggaggtaagtttatatggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataatttaaggaggttaagaggatgaatatagttgtttgtttaaaacaagttccagatacagcggaagttagaatagatccagttaagggaacacttataagagaaggagttccatcaataataaatccagatgataaaaacgcacttgaggaagctttagtattaaaagataattatggtgcacatgtaacagttataagtatgggacctccacaagctaaaaatgctttagtagaagctttggctatgggtgctgatgaagctgtacttttaacagatagagcatttggaggagcagatacacttgcgacttcacatacaattgcagcaggaattaagaagctaaaatatgatatagtttttgctggaaggcaggctatagatggagatacagctcaggttggaccagaaatagctgagcatcttggaatacctcaagtaacttatgttgagaaagttgaagttgatggagatactttaaagattagaaaagcttgggaagatggatatgaagttgttgaagttaagacaccagttcttttaacagcaattaaagaattaaatgttccaagatatatgagtgtagaaaaaatattcggagcatttgataaagaagtaaaaatgtggactgccgatgatatagatgtagataaggctaatttaggtcttaaaggttcaccaactaaagttaagaagtcatcaactaaagaagttaaaggacagggagaagttattgataagcctgttaaggaagcagctgatatgttgtctcaaaattaaaagaagaacacatatttaagttaggagggatttttcaatgaataaagcagattacaagggcgtatgggtgtttgctgaacaaagagacggagaattacaaaaggtatcattggaattattaggtaaaggtaaggaaatggctgagaaattaggcgttgaattaacagctgttttacttggacataatactgaaaaaatgtcaaaggatttattatctcatggagcagataaggttttagcagcagataatgaacttttagcacatttttcaacagatggatatgctaaagttatatgtgatttagttaatgaaagaaagccagaaatattattcataggagctactttcataggaagagatttaggaccaagaatagcagcaagactttctactggtttaactgctgattgtacatcacttgacatagatgtagaaaatagagatttattggctacaagaccagcgtttggtggaaatttgatagctacaatagtttgttcagaccacagaccacaaatggctacagtaagacctggtgtgttttttgaaaaattacctgttaatgatgcaaatgtttctgatgataaaatagaaaaagttgcaattaaattaacagcatcagacataagaacaaaagtttcaaaagttgttaagcttgctaaagatattgcagatatcggagaagctaaggtattagttgctggtggtagaggagttggaagcaaagaaaactttgaaaaacttgaagagttagcaagtttacttggtggaacaatagccgcttcaagagcagcaatagaaaaagaatgggttgataaggaccttcaagtaggtcaaactggtaaaactgtaagaccaactctttatattgcatgtggtatatcaggagctatccagcatttagcaggtatgcaagattcagattacataattgctataaataaagatgtagaagccccaataatgaaggtagcagatttggctatagttggtgatgtaaataaagttgtaccagaattaatagctcaagttaaagctgctaataattaagataaataaaaagaattatttaaagcttattatgccaaaatacttatatagtattttggtgtaaatgcattgatagtttctttaaatttagggaggtctgtttaatgcattgatagttctttaaatttagggaggtctgtttaatgaaaaaggtatgtgttataggtgcaggtactatgggttcaggaattgctcaggcatttgcagctaaaggatttgaagtagtattaagagatattaaagatgaatttgttg

SEQ ID NO: 6 - DNA, 2104 bp, Escherichia coli
gatcgaattcaaagtcggcccagaagaaaaggactggagcatggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataagccatgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaaccgctcaaggcttgaacaacatcgcgcttatcgttaaggtaagcgcgtattttttttacccgccaggacaactcgaggatc

SEQ ID NO: 7 - DNA, 6037 bp, artificial sequence
  Plasmid comprising ilv N/B from E. coli
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcggcttgatcgaattcaaagtcggcccagaagaaaaggactggagcatggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataagccatgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaaccgctcaaggcttgaacaacatcgcgcttatcgttaaggtaagcgcgtattttttttacccgccaggacaactcgaggatcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagtagc

SEQ ID NO: 8 - DNA, 3645 bp, Pseudomonas putida
gatcgaattcaattgaaaaaggaagagtatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcgaagcttgatc

SEQ ID NO: 9 - DNA, 7917 bp, artificial sequence
  Plasmid comprising bkd A1, A2, B from P. putida
atgaccatgattacgccaagcgcgcaattaaccctcactaaagggaacaaaagctgggtaccgggccccccctcgaggtcgacggtatcgataagcttgatatccactgtggaattcgcccttgatcgaattcaattgaaaaaggaagagtatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcgaagcttgatcaagggcgaattccacattggtcgctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattagtgtccttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 10 - DNA, 4723 bp, artificial sequence
  Plasmid sequence comprising multiple cloning sequence.
gaattcgcattaagcttgcactcgagcgtcgaccgttctagtttaaacatattctgaaatgagctgttgacaattaatcatcggctcgtataatgtgtggaattgtgagcggataacaatttcacacacatctagacgcgatatccgaatcccgggcttcgtgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 11 - DNA, 2792 bp, artificial sequence
  Plasmid comprising multiple cloning sequence.
gagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgaattcgcattaagcttgcactcgagcgtcgaccgttctagacgcgatatccgaatcccgggcttcgtgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttattttttgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 12 - DNA, 7031 bp, artificial sequence
  Plasmid comprising adhe from C. acetobutylicum.
atgaccatgattacgccaagcgcgcaattaaccctcactaaagggaacaaaagctgggtaccgggccccccctcgaggtcgacggtatcgataagcttgatatccactgtggaattcgcccttctctcccgggtataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagacgcggccgcagagaagggcgaattccacattggtcgctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattagtgtccttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 13 - DNA, 7917 bp, artificial sequence; identical to SEQ ID NO: 9
  Plasmid comprising BKD A1, A2, B from P. putida.

SEQ ID NO: 14 - DNA, 3933 bp, artificial sequence
  commercial plasmid.
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcggcttaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagtagc

SEQ ID NO: 15 - DNA, 1536 bp, Escherichia coli
gatcgtcgacataagaagcacaacatcacgaggaatcaccatggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaagtgcgcgctgtctagagatc

SEQ ID NO: 16 - DNA, 5469 bp, artificial sequence
  Plasmid comprising ilvC gene sequence from E. coli.
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgccttgatcgtcgacataagaagcacaacatcacgaggaatcaccatggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaagtgcgcgctgtctagagatcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagtagc

SEQ ID NO: 17 - DNA, 1933 bp, Escherichia coli
gatctctagaccgtcccatttacgagacagacactgggagtaaataaagtatgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaataatggctgactcgcaacccgcggccgcgatc

SEQ ID NO: 18 - DNA, 5866 bp, artificial sequence
  Plasmid comprising ilvD gene sequence from E. coli.
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgccttgatctctagaccgtcccatttacgagacagacactgggagtaaataaagtatgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaataatggctgactcgcaacccgcggccgcgatcaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagtagc

SEQ ID NO: 19 - DNA, 5142 bp, artificial sequence
  Plasmid comprising multiple cloning site.
gaattcgcattaagcttgcactcgagcgtcgaccgttctagacgcgatatsmaccgaatcccgggcttcgtgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtamramtgagtattcaacatttccgtgtcgcccttattcccttttttgcggcatttshrvaaaramtgccttcctgtttttgctcacccagaaacgctggtgaaagtaaaagatgccvahtvkvkdaramtgaagatcagttgggtgcacgagtgggttacatcgaactggatctcaacadgarvgydnsramgcggtaagatccttgagagttttcgccccgaagaacgttttccaatgatggksrrmmramagcacttttaaagttctgctatgtggcgcggtattatcccgtgttgacgcstkvcgavsrvdaramcgggcaagagcaactcggtcgccgcatacactattctcagaatgacttggggrrhysndvramttgagtactcaccagtcacagaaaagcatcttacggatggcatgacagtaysvtkhtdgmtvramagagaattatgcagtgctgccataaccatgagtgataacactgcggccaarcsaatmsdntaanramcttacttctgacaacgatcggaggaccgaaggagctaaccgcttttttgcttggktahramacaacatgggggatcatgtaactcgccttgatcgttgggaaccggagctgnmgdhvtrdrwramaatgaagccataccaaacgacgagcgtgacaccacgatgctgtagcaatgnandrdttmramgcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccattstarswramgcaccgcgacgcaacgcggggaggcagacaaggtatagggcggcgcctacagrracvyaagvramaatccatgccaacccgttccatgtgctcgccgaggcggcataaatcgccgwagnwtsasaayaramtgacgatcagcggtccagtgatcgaagttaggctggtaagagccgcgagctvgtststaaramgatccttgaagctgtccctgatggtcgtcatctacctgcctggacagcatmvvyghramsgghdddvrsmramggcctgcaacgcgggcatcccgatgccgccggaagcgagaagaatcataagrghdaagsknhnramaamgggsamramtggggaaggccatccagcctcgcgtcgcgaacgccagcaagacgtagcccgghasrrrdvaramamwgrtaavygramagcgcgtcggccgccatgccggcgataatggcctgcttctcgccgaaacgrvgrhagdngatramadaamgaakgrramtttggtggcgggaccagtgacgaaggcttgagcgagggcgtgcaagattcgggtsdgsgvdsramktagtvaaahramcgaataccgcaagcgacaggccgatcatcgtcgcgctccagcgaaagcggyrkradhrraakavramgvasgmtaswrrramtcctcgccgaaaatgacccagagcgctgccggcacctgtcctacgagttgandrcrhsyramdgvwaavgvramcatgataaagaagacagtcataagtgcggcgacgatagtcatgccccgcghdkdshkcgddsharrammvtmaavtmgrramcccaccggaaggagctgactgggttgaaggctctcaagggcatcggtcgagadwvgsghrstramawrssvnarmrramcgctctcccttatgcgactcctgcattaggaagcagcccagtagtaggttsmrhramrgkhsmcgnramgaggccgttgagcaccgccgccgcaaggaatggtgcatgcaaggagatgggnvaaaahsramcgcccaacagtcccccggccacggggcctgccaccatacccacgccgaaaagggavgavmgvgramcaagcgctcatgagcccgaagtggcgagcccgatcttccccatcggtgatcasmramgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 20 - DNA, 8343 bp, artificial sequence
  Plasmid comprising bkd A1, A2, B from P. putida
gaattcaattgaaaaaggaagagtatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcgaagcttgcactcgagcgtcgaccgttctagtttaaacatattctgaaatgagctgttgacaattaatcatcggctcgtataatgtgtggaattgtgagcggataacaatttcacacacatctagacgcgatatccgaatcccgggcttcgtgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 21 - DNA, 11493 bp, artificial sequence
  Plasmid comprising bkd A1, A2, B gene sequence from P. putida and adhe gene sequence from C. acetobutylicum.
gaattcaattgaaaaaggaagagtatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcgaagcttgcactcgagcgtcgaccgttctagacgcgatatsmaccgaatcccgggtataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagacgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtamramtgagtattcaacatttccgtgtcgcccttattcccttttttgcggcatttshrvaaaramtgccttcctgtttttgctcacccagaaacgctggtgaaagtaaaagatgccvahtvkvkdaramtgaagatcagttgggtgcacgagtgggttacatcgaactggatctcaacadgarvgydnsramgcggtaagatccttgagagttttcgccccgaagaacgttttccaatgatggksrrmmramagcacttttaaagttctgctatgtggcgcggtattatcccgtgttgacgcstkvcgavsrvdaramcgggcaagagcaactcggtcgccgcatacactattctcagaatgacttggggrrhysndvramttgagtactcaccagtcacagaaaagcatcttacggatggcatgacagtaysvtkhtdgmtvramagagaattatgcagtgctgccataaccatgagtgataacactgcggccaarcsaatmsdntaanramcttacttctgacaacgatcggaggaccgaaggagctaaccgcttttttgcttggktahramacaacatgggggatcatgtaactcgccttgatcgttgggaaccggagctgnmgdhvtrdrwramaatgaagccataccaaacgacgagcgtgacaccacgatgctgtagcaatgnandrdttmramgcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccattstarswramgcaccgcgacgcaacgcggggaggcagacaaggtatagggcggcgcctacagrracvyaagvramaatccatgccaacccgttccatgtgctcgccgaggcggcataaatcgccgwagnwtsasaayaramtgacgatcagcggtccagtgatcgaagttaggctggtaagagccgcgagctvgtststaaramgatccttgaagctgtccctgatggtcgtcatctacctgcctggacagcatmvvyghramsgghdddvrsmramggcctgcaacgcgggcatcccgatgccgccggaagcgagaagaatcataagrghdaagsknhnramaamgggsamramtggggaaggccatccagcctcgcgtcgcgaacgccagcaagacgtagcccgghasrrrdvaramamwgrtaavygramagcgcgtcggccgccatgccggcgataatggcctgcttctcgccgaaacgrvgrhagdngatramadaamgaakgrramtttggtggcgggaccagtgacgaaggcttgagcgagggcgtgcaagattcgggtsdgsgvdsramktagtvaaahramcgaataccgcaagcgacaggccgatcatcgtcgcgctccagcgaaagcggyrkradhrraakavramgvasgmtaswrrramtcctcgccgaaaatgacccagagcgctgccggcacctgtcctacgagttgandrcrhsyramdgvwaavgvramcatgataaagaagacagtcataagtgcggcgacgatagtcatgccccgcghdkdshkcgddsharrammvtmaavtmgrramcccaccggaaggagctgactgggttgaaggctctcaagggcatcggtcgagadwvgsghrstramawrssvnarmrramcgctctcccttatgcgactcctgcattaggaagcagcccagtagtaggttsmrhramrgkhsmcgnramgaggccgttgagcaccgccgccgcaaggaatggtgcatgcaaggagatgggnvaaaahsramcgcccaacagtcccccggccacggggcctgccaccatacccacgccgaaaagggavgavmgvgramcaagcgctcatgagcccgaagtggcgagcccgatcttccccatcggtgatcasmramgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 22 - DNA, 75 bp, artificial sequence
  oligonucleotide primer
agctgcggccgcacgaagcccgggattcggatatcgcgtctagaacggtcgacgctcgagtgcaagcttaatgcg

SEQ ID NO: 23 - DNA, 13706 bp, artificial sequence
  Plasmid comprising icmA, B gene sequence from S. avermitilis and bkdA1, A2, B gene sequence from P. Putida and adhe gene sequence from C. acetobutylicum.
agaattcaattgaaaaaggaagagtatgaacgagtacgcccccctgcgtttgcatgtgcccgagcccaccggccggccaggctgccagaccgatttttcctacctgcgcctgaacgatgcaggtcaagcccgtaaaccccctgtcgatgtcgacgctgccgacaccgccgacctgtcctacagcctggtccgcgtgctcgacgagcaaggcgacgcccaaggcccgtgggctgaagacatcgacccgcagatcctgcgccaaggcatgcgcgccatgctcaagacgcggatcttcgacagccgcatggtggttgcccagcgccagaagaagatgtccttctacatgcagagcctgggcgaagaagccatcggcagcggccaggcgctggcgcttaaccgcaccgacatgtgcttccccacctaccgtcagcaaagcatcctgatggcccgcgacgtgtcgctggtggagatgatctgccagttgctgtccaacgaacgcgaccccctcaagggccgccagctgccgatcatgtactcggtacgcgaggccggcttcttcaccatcagcggcaacctggcgacccagttcgtgcaggcggtcggctgggccatggcctcggcgatcaagggcgataccaagattgcctcggcctggatcggcgacggcgccactgccgaatcggacttccacaccgccctcacctttgcccacgtttaccgcgccccggtgatcctcaacgtggtcaacaaccagtgggccatctcaaccttccaggccatcgccggtggcgagtcgaccaccttcgccggccgtggcgtgggctgcggcatcgcttcgctgcgggtggacggcaacgacttcgtcgccgtttacgccgcttcgcgctgggctgccgaacgtgcccgccgtggtttgggcccgagcctgatcgagtgggtcacctaccgtgccggcccgcactcgacctcggacgacccgtccaagtaccgccctgccgatgactggagccacttcccgctgggtgacccgatcgcccgcctgaagcagcacctgatcaagatcggccactggtccgaagaagaacaccaggccaccacggccgagttcgaagcggccgtgattgctgcgcaaaaagaagccgagcagtacggcaccctggccaacggtcacatcccgagcgccgcctcgatgttcgaggacgtgtacaaggagatgcccgaccacctgcgccgccaacgccaggaactgggggtttgagatgaacgaccacaacaacagcatcaacccggaaaccgccatggccaccactaccatgaccatgatccaggccctgcgctcggccatggatgtcatgcttgagcgcgacgacaatgtggtggtgtacggccaggacgtcggctacttcggcggcgtgttccgctgcaccgaaggcctgcagaccaagtacggcaagtcccgcgtgttcgacgcgcccatctctgaaagcggcatcgtcggcaccgccgtgggcatgggtgcctacggcctgcgcccggtggtggaaatccagttcgctgactacttctacccggcctccgaccagatcgtttctgaaatggcccgcctgcgctaccgttcggccggcgagttcatcgccccgctgaccctgcgtatgccctgcggtggcggtatctatggcggccagacacacagccagagcccggaagcgatgttcactcaggtgtgcggcctgcgcaccgtaatgccatccaacccgtacgacgccaaaggcctgctgattgcctcgatcgaatgcgacgacccggtgatcttcctggagcccaagcgcctgtacaacggcccgttcgacggccaccatgaccgcccggttacgccgtggtcgaaacacccgcacagcgccgtgcccgatggctactacaccgtgccactggacaaggccgccatcacccgccccggcaatgacgtgagcgtgctcacctatggcaccaccgtgtacgtggcccaggtggccgccgaagaaagtggcgtggatgccgaagtgatcgacctgcgcagcctgtggccgctagacctggacaccatcgtcgagtcggtgaaaaagaccggccgttgcgtggtagtacacgaggccacccgtacttgtggctttggcgcagaactggtgtcgctggtgcaggagcactgcttccaccacctggaggcgccgatcgagcgcgtcaccggttgggacaccccctaccctcacgcgcaggaatgggcttacttcccagggccttcgcgggtaggtgcggcattgaaaaaggtcatggaggtctgaatgggcacgcacgtcatcaagatgccggacattggcgaaggcatcgcgcaggtcgaattggtggaatggttcgtcaaggtgggcgacatcatcgccgaggaccaagtggtagccgacgtcatgaccgacaaggccaccgtggaaatcccgtcgccggtcagcggcaaggtgctggccctgggtggccagccaggtgaagtgatggcggtcggcagtgagctgatccgcatcgaagtggaaggcagcggcaaccatgtggatgtgccgcaagccaagccggccgaagtgcctgcggcaccggtagccgctaaacctgaaccacagaaagacgttaaaccggcggcgtaccaggcgtcagccagccacgaggcagcgcccatcgtgccgcgccagccgggcgacaagccgctggcctcgccggcggtgcgcaaacgcgccctcgatgccggcatcgaattgcgttatgtgcacggcagcggcccggccgggcgcatcctgcacgaagacctcgacgcgttcatgagcaaaccgcaaagcgctgccgggcaaacccccaatggctatgccaggcgcaccgacagcgagcaggtgccggtgatcggcctgcgccgcaagatcgcccagcgcatgcaggacgccaagcgccgggtcgcgcacttcagctatgtggaagaaatcgacgtcaccgccctggaagccctgcgccagcagctcaacagcaagcacggcgacagccgcggcaagctgacactgctgccgttcctggtgcgcgccctggtcgtggcactgcgtgacttcccgcagataaacgccacctacgatgacgaagcgcagatcatcacccgccatggcgcggtgcatgtgggcatcgccacccaaggtgacaacggcctgatggtacccgtgctgcgccacgccgaagcgggcagcctgtgggccaatgccggtgagatttcacgcctggccaacgctgcgcgcaacaacaaggccagccgcgaagagctgtccggttcgaccattaccctgaccagcctcggcgccctgggcggcatcgtcagcacgccggtggtcaacaccccggaagtggcgatcgtcggtgtcaaccgcatggttgagcggcccgtggtgatcgacggccagatcgtcgtgcgcaagatgatgaacctgtccagctcgttcgaccaccgcgtggtcgatggcatggacgccgccctgttcatccaggccgtgcgtggcctgctcgaacaacccgcctgcctgttcgtggagtgagcatgcaacagactatccagacaaccctgttgatcatcggcgaagcttgcactcgagcgtcgaccgttctagctactactgcaagtcgagaattctccgtcgactgactgagtcagtcggatcctatcggtacatggaggcagtgatgggcgtggcggccggtccgattcgtgttgtagttgcaaaaccggggctggatggtcatgaccgcggtgctaaggtaatcgcgcgtgcactgcgtgacgctggcatggaagtaatttataccggcctgcaccagacgccggagcaagtggtcgacactgctatccaagaggacgctgatgccattggcctgagcatcctgtctggggcacataatacactgtttgctcgcgtgctggagctgctgaaagaacgcgatgcagaagatattaaagttttcggcgggggcatcattccggaggccgacatcgcaccgctgaaggaaaaaggtgtcgctgagatttttaccccgggcgcgacgactacaagtatcgtggaatgggtacgtggtaacgttcgccaagccgtgtaatagcatcgactttcgagattttcaggagctaaggaagctaaaatggacgcggacgccattgaagagggccgtcgccgttggcaggcgcgttacgataaagctcgcaagcgcgacgcggacttcaccacgctgtcgggcgatccggtagatccggtttacgggccacgcccaggcgatacttacgacggcttcgaacgtattgggtggccgggtgagtacccgtttactcgtggactgtacgccactggctaccgcggtcgtacatggactatccgccaattcgcagggtttggcaacgccgaacagaccaacgaacgttataaaatgattctggcgaacggtggcggaggtctgtcagtagccttcgatatgccgacgctgatgggtcgtgatagtgacgacccacgttctctgggagaagtagggcactgcggcgtggcaattgattccgctgcggatatggaagtgctgttcaaagatattccgctgggcgacgtcaccacgtcgatgacaattagcggcccggccgtaccagtattctgcatgtatctggtagctgcggaacgccaaggtgtcgaccctgcagtcctgaacgggacactgcagacggacatctttaaggaatatattgcccagaaagaatggctgttccaaccggagcctcaccttcgtctgatcggtgatctgatggaacactgcgcacgcgatattccggcgtataaacctctgagcgttagtggttaccacatccgtgaggcaggagccacagcagcgcaggaactggcttatactctggccgatggttttgggtacgttgaactgggcctgtcacgcggcctggacgttgatgtattcgctccgggcctgtcgttcttttttgacgcgcatgttgatttcttcgaggagatcgcgaaatttcgtgccgcacgccgtatctgggcgcgctggctgcgtgacgagtatggtgctaagacagaaaaagcacaatggctgcgcttccacacgcaaactgctggtgtaagcctgacagcacaacaaccgtataacaacgtggtccgtactgccgttgaagcgctggctgcagttctggggggtactaatagcctgcatacgaacgccctggacgagacactggccctgccgtctgaacaggctgcagagatcgctctgcgcactcaacaggtactgatggaggaaacaggggttgcgaacgtagctgacccgctgggcggaagctggtatatcgagcaactgaccgaccgtatcgaagcggatgccgaaaagattttcgagcaaatccgcgaacgcgggcgtcgtgcttgccctgatggacaacacccaattggcccgatcacctcagggattctgcgcggtatcgaggacgggtggtttacgggggaaattgctgagagcgcttttcaataccagcgttcgctggagaaaggtgataaacgcgtggtaggcgttaactgcctggaaggtagcgtgacgggggacctggagatcctgcgcgtatctcacgaagttgagcgcgaacaggtgcgtgaactggccggccgcaagggtcgtcgcgatgacgcgcgtgtacgcgctagtctggatgcaatgctggccgcggctcgtgatggctcaaatatgattgcacctatgctggaggccgtccgcgcggaagcaaccctgggggaaatctgcggggttctgcgtgatgaatggggcgtttatgtggagccgccgggtttttaataggatgggtataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagacgcggccgcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtamramtgagtattcaacatttccgtgtcgcccttattcccttttttgcggcatttshrvaaaramtgccttcctgtttttgctcacccagaaacgctggtgaaagtaaaagatgccvahtvkvkdaramtgaagatcagttgggtgcacgagtgggttacatcgaactggatctcaacadgarvgydnsramgcggtaagatccttgagagttttcgccccgaagaacgttttccaatgatggksrrmmramagcacttttaaagttctgctatgtggcgcggtattatcccgtgttgacgcstkvcgavsrvdaramcgggcaagagcaactcggtcgccgcatacactattctcagaatgacttggggrrhysndvramttgagtactcaccagtcacagaaaagcatcttacggatggcatgacagtaysvtkhtdgmtvramagagaattatgcagtgctgccataaccatgagtgataacactgcggccaarcsaatmsdntaanramcttacttctgacaacgatcggaggaccgaaggagctaaccgcttttttgcttggktahramacaacatgggggatcatgtaactcgccttgatcgttgggaaccggagctgnmgdhvtrdrwramaatgaagccataccaaacgacgagcgtgacaccacgatgctgtagcaatgnandrdttmramgcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccattstarswramgcaccgcgacgcaacgcggggaggcagacaaggtatagggcggcgcctacagrracvyaagvramaatccatgccaacccgttccatgtgctcgccgaggcggcataaatcgccgwagnwtsasaayaramtgacgatcagcggtccagtgatcgaagttaggctggtaagagccgcgagctvgtststaaramgatccttgaagctgtccctgatggtcgtcatctacctgcctggacagcatmvvyghramsgghdddvrsmramggcctgcaacgcgggcatcccgatgccgccggaagcgagaagaatcataagrghdaagsknhnramaamgggsamramtggggaaggccatccagcctcgcgtcgcgaacgccagcaagacgtagcccgghasrrrdvaramamwgrtaavygramagcgcgtcggccgccatgccggcgataatggcctgcttctcgccgaaacgrvgrhagdngatramadaamgaakgrramtttggtggcgggaccagtgacgaaggcttgagcgagggcgtgcaagattcgggtsdgsgvdsramktagtvaaahramcgaataccgcaagcgacaggccgatcatcgtcgcgctccagcgaaagcggyrkradhrraakavramgvasgmtaswrrramtcctcgccgaaaatgacccagagcgctgccggcacctgtcctacgagttgandrcrhsyramdgvwaavgvramcatgataaagaagacagtcataagtgcggcgacgatagtcatgccccgcghdkdshkcgddsharrammvtmaavtmgrramcccaccggaaggagctgactgggttgaaggctctcaagggcatcggtcgagadwvgsghrstramawrssvnarmrramcgctctcccttatgcgactcctgcattaggaagcagcccagtagtaggttsmrhramrgkhsmcgnramgaggccgttgagcaccgccgccgcaaggaatggtgcatgcaaggagatgggnvaaaahsramcgcccaacagtcccccggccacggggcctgccaccatacccacgccgaaaagggavgavmgvgramcaagcgctcatgagcccgaagtggcgagcccgatcttccccatcggtgatcasmramgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 24 - DNA, 1311 bp, Homo sapiens
agtccgggagattctcgctctgctgctttagtttcggagtgtttggcgacggggcagcgcgagatgtggaggctcatgtcgaggtttaatgcattcaaaaggactaataccatactgcaccatttgagaatgtccaagcacacagatgcagcagaagaggtgctattggaaaaaaaaggttgcgcgggagtcataacactaaacagaccaaagttcctcaatgcactgactcttaatatgattcggcagatttatccacagctaaagaagtgggaacaagatcctgaaactttcgtgatcattataaagggagcaggaggaaaggctttctgtgccgggggtgatatcagagtgatctcggaagctgaaaaggcaaaacagaagatagctccagttttcttcagagaagaatatatgctgaataatgctgttggttcttgccagaaaccttatgttgcacttattcatggaattacaatgggtgggggagttggtctctcagtccatgggcaatttcgagtggctacagaaaagtgtctttttgctatgccagaaactgcaataggactgttccctgatgtgggtggaggttatttctttgccacgactccaaggaaaacttggttacttccttgcattaacggattcagactaaaaggaagagatgtgtacagagcaggaattgctacacactttgtagattctgaaaagttggccatgttagaggaagatttgttagccttgaaatctccttcaaaagaaaatattgcatctgtcttagaaaattaccatacagagtctaagattgatcgagacaagtcttttatacttgaggaacacatggacaaaataaacagttgtttttcagccaatactgtggaagaaattattgaaaacttacagcaagatggttcatcttttgccctagagcaattgaaggtaattaataaaatgtctccaacatctctaaagatcacactaaggcaactcatggaggggtcttcaaagaccttgcaagaagtactaactatggagtatcggctaagtcaagcttgtatgagaggtcatgactttcatgaaggcgttagagctgttttaattgataaagaccagagtccaaaatggaaaccagctgatctaaaagaagttactgaggaagatttgaataatcactttaagtctttgggaagcagtgatttgaaattttgaggtgacaggcttttaaggtatattttgtagcatgggttggcaatctacagcatgtgggccaaatccagcctgctgcctgtttttatataccctgtaagcaag

SEQ ID NO: 25 - DNA, 1083 bp, Saccharomyces cerevisiae
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 26 - DNA, 30 bp, artificial sequence
  oligonucleotide primer
ctctcccgggtataaggcatcaaagtgtgt

SEQ ID NO: 27 - DNA, 36 bp, artificial sequence
  oglioneucleotide primer
ctctcccgggctcgaggtctatgtgcttcatgaagc

SEQ ID NO: 28 - DNA, 52 bp, artificial sequence
  oglionucleotide primer
gatcgaattcaattgaaaaaggaagagtatgaacgagtacgcgccccttgcg

SEQ ID NO: 29 - DNA, 33 bp, artificial sequence
  oligonucleotide primer
gatcaagcttcgccgatgatcaacagggttgtc

SEQ ID NO: 30 - DNA, 28 bp, artificial sequence
  oligonucleotide primer
atcccggggaggagtaaaacatgagaga

SEQ ID NO: 31 - DNA, 34 bp, artificial sequence
  oligonucleotide primer
atcccgggctcgagttagtctctttcaactacga

SEQ ID NO: 32 - DNA, 44 bp, artificial sequence
  oligonucleotide primer
atcccgggatattttaggaggattagtcatggaactaaacaatg

SEQ ID NO: 33 - DNA, 46 bp, artificial sequence
  oligonucleotide primer
atcccgggagatcttgtaaacttattttgaataatcgtagaaaccc

SEQ ID NO: 34 - DNA, 60 bp, artificial sequence
  oligonucleotide primer
gatcgaattcaaagtcggcccagaagaaaaggactggagcatggcaagttcgggcacaac

SEQ ID NO: 35 - DNA, 70 bp, artificial sequence
  oligonucleotide primer
gatcctcgagtgtcctggcgggtaaaaaaaatacgcgcttaccttaacgataagcgcgatgttgttcaag

SEQ ID NO: 36 - DNA, 60 bp, artificial sequence
  oligonucleotide primer
gatctctagacagcgcgcacttaacccgcaacagcaatacgtttcatatctgtcatatag

SEQ ID NO: 37 - DNA, 60 bp, artificial sequence; identical to SEQ ID NO: 36
  oligonucleotide primer

SEQ ID NO: 38 - DNA, 50 bp, artificial sequence
  oligonucleotide primer
gatctctagaccgtcccatttacgagacagacactgggagtaaataaagt

SEQ ID NO: 39 - DNA, 52 bp, artificial sequence
  oligonucleotide primer
gatcgcggccgcgggttgcgagtcagccattattaaccccccagtttcgatt

SEQ ID NO: 40 - DNA, 75 bp, artificial sequence
  oligonucleotide primer
aattcgcattaagcttgcactcgagcgtcgaccgttctagacgcgatatccgaatcccgggcttcgtgcggccgc

SEQ ID NO: 41 - DNA, 75 bp, artificial sequence; identical to SEQ ID NO: 22
  oligonucleotide primer

SEQ ID NO: 42 - DNA, 60 bp, artificial sequence
  oligonucleotide primer
ctagtttaaacatattctgaaatgagctgttgacaattaatcatcggctcgtataatgtg

SEQ ID NO: 43 - DNA, 34 bp, artificial sequence
  oligonucleotide primer
tggaattgtgagcggataacaatttcacacacat

SEQ ID NO: 44 - DNA, 58 bp, artificial sequence
  oligonucleotide primer
ctagatgtgtgtgaaattgttatccgctcacaattccacacattatacgagccgatga

SEQ ID NO: 45 - DNA, 36 bp, artificial sequence
  oligonucleotide primer
ttaattgtcaacagctcatttcagaatatgtttaaa

SEQ ID NO: 46 - DNA, 14 bp, artificial sequence
  oligonucleotide primer
gagcgtcagacccc

SEQ ID NO: 47 - DNA, 18 bp, artificial sequence
  oligonucleotide primer
gtcaagtcagcgtaatgc

SEQ ID NO: 48 - DNA, 17 bp, artificial sequence
  oligonucleotide primer
tgcaccaatgcttctgg

SEQ ID NO: 49 - DNA, 31 bp, artificial sequence
  oligonucleotide primer
gaaaaataaacaaaagagtttgtagaaacgc

SEQ ID NO: 50 - DNA, 75 bp, artificial sequence; identical to SEQ ID NO: 40
  oligonucleotide primer

(19) United States
(12) Patent Application Publication
(10) Pub. No.: US 2011/0281314 A1
(43) Pub. Date: Nov. 17, 2011

(54) METHODS, SYSTEMS AND COMPOSITIONS RELATED TO MICROBIAL BIO-PRODUCTION OF BUTANOL AND/OR ISOBUTANOL

(76) Inventor: Michael D. Lynch, Boulder, CO (US)

(21) Appl. No.: 13/057,359

(22) PCT Filed: Aug. 4, 2009
(86) PCT No.: PCT/US2009/052748

§ 371 Date: Jul. 28, 2011

Related U.S. Application Data
(60) Provisional application No. 61/085,986, filed on Aug. 4, 2008.

(51) Int. Cl. C12P 7/16; C12N 1/15; C12N 1/13; C12N 1/19; C12M 1/00; C12N 1/21

(52) U.S. Cl. 435/160; 435/289.1; 435/252.3; 435/252.34; 435/252.31; 435/252.32; 435/252.33; 435/257.2; 435/254.23; 435/254.22; 435/254.21; 435/254.11

(57) ABSTRACT

Embodiments herein generally relate to methods, compositions, systems and uses for enabling bio-production of or increasing bio-production of alcohol molecules by microorganisms. Certain embodiments relate to compositions and methods enabling or increasing the bio-production of 4-carbon alcohol molecules by bacteria. In some embodiments, compositions and methods relate to introducing isobutyryl-CoA isomerase to a culture of microorganisms to enable or increase the bio-production of four-carbon alcohols. Variations of biosynthesis pathways for microbial bio-production of butanol and/or isobutanol are provided.

### RELATED APPLICATIONS

This application claims priority to the following U.S. Provisional patent application: 61/085,986, filed Aug. 4, 2008.

### STATEMENT REGARDING FEDERALLY SPONSORED RESEARCH

N/A

### REFERENCE TO A SEQUENCE LISTING

This provisional patent application provides a paper copy of sequence listings that are to be provided on compact disk in appropriate format in a later submission.

### FIELD OF THE INVENTION

The present invention relates to methods, compositions and systems for enabling or increasing the production of alcohol compounds by microorganisms, and more particularly to the making of and the use of recombinant microorganisms that bio-produce butanol and/or isobutanol, such as in industrial systems based on directed microbial biosynthetic activity.

### BACKGROUND

Four carbon alcohols derived from biological fermentations are of much industrial interest. The interest in these alcohols primarily stems from their potential use as fuels available from renewable resources, and also from their current uses, including as solvents.

Oil costs have risen dramatically over the past several years. Most experts now believe that such cost increases will continue and that oil production capacity will peak in the near future. Alternative sources of inexpensive materials and energy for the production of fuels and other chemicals must be developed. Bio-production, such as by microbial biosynthetic processes, seeks to utilize renewable resources, such as agricultural or municipal waste, to provide substantially non-petroleum-based fuels and other chemicals. The basic model involves the conversion of agricultural high-cellulose materials (e.g., cellulosic grasses and materials), waste material (e.g., food and industrial fermentation byproducts), and/or agricultural primary products (e.g. corn) into sugars (e.g. hexoses, pentoses) that can be enzymatically converted by bioengineered organisms to produce value added products such as fuels (e.g., ethanol or hydrogen) or commodity chemicals (e.g. monomers/polymers). While much debate still exists regarding the long term commercial viability of ethanol as a gasoline replacement, biological routes for the production of commodity chemicals have been proven as economically attractive alternatives to conventional petrochemical routes. As one example, a decade-long DuPont/Genencor collaboration led DuPont into investing in the development of an 800,000 liters E. coli based process for the production of 1,3 propanediol (an estimated $5-8 billion/year product).

Reflective of the interest to utilize bio-production approaches to produce butanol and isobutanol are a number of references directed to various aspects of such bio-production, including the following patents and patent applications, which are incorporated by reference herein for their respective teachings of natural and recombinant biosynthetic pathways directed to production of various C-4 alcohols: U.S. Pat. No. 5,192,673; U.S. Pat. No. 6,358,717; PCT Publication No. WO 2007/050671; PCT Publication No. WO2007/041269; PCT Publication No. WO2007/089677; U.S. Publication No. 2007/0092957; and U.S. Publication No. 2007/0292927.

Notwithstanding the above, there remains a need in the art for novel methods, systems and compositions related to microbial production of butanol and isobutanol, particularly where these are efficient and effective to produce such alcohols in large quantities, for example, for use as biofuels.

### SUMMARY OF THE INVENTION

The present invention includes a genetically modified microorganism (such as a recombinant microorganism), comprising genetic elements any of the butanol and/or isobutanol biosynthesis pathway alternatives described herein, and a method of butanol and/or isobutanol bio-production that utilizes any such genetically modified microorganism.

In one aspect of the invention, such microorganism comprises an enzyme that catalyzes the reaction between butyryl-CoA and isobutyrl-CoA (e.g., an isobutyryl-CoA mutase, e.g, S. avertmitilis icmA,B), wherein that microorganism is able to produce butanol (or in related aspects, isobutanol, or both butanol and isobutanol). This enzymatic conversion step is referred to as the ‘bridge’ herein.

Thus, a recombinant microorganism according to the present invention may comprise genetic elements encoding enzymes that catalyze enzymatic conversion steps of any of the butanol and/or isobutanol production pathway alternatives described and/or taught herein, in various embodiments including the ‘bridge’, to provide a recombinant microorganism that produces butanol and/or isobutanol. Such recombinant microorganism may demonstrate increased productivity and yield of butanol and/or isobutanol (compared with a non-modified control microorganism). Various embodiments of the invention may comprise any combination of the alternative approaches described herein, and depicted in FIG. 1, for the bio-production of butanol and/or isobutanol.

In related aspects, genetic modifications are provided to reduce or eliminate bio-production of undesired metabolic products, and/or mutant strains such as exemplified above by NZN111 and JW1375, may also be used in combination with genetic modifications directed to production of butanol and/or isobutanol.

In further aspects, any such microorganism further comprises one or more genetic modifications providing increased tolerance to butanol and/or isobutanol. Standard selection methods may be used to identify a more tolerant organism (into which nucleic acid sequences for production pathways may be introduced), and/or analysis of data obtained from the Gill et al. technique, discussed herein, or from other known techniques, to identify genetic elements related to increased tolerance. These genetic elements may be introduced into a microorganism, along with genetic elements to provide and/or improve one or more of the butanol/isobutanol production pathway alternatives.

### BRIEF DESCRIPTION OF THE FIGURES AND SEQUENCE DESCRIPTIONS

The various aspects of the present invention may be more fully understood from the following figures and sequence descriptions, which form part of this application.

FIG. 1 provides a summary of two metabolic pathways that are joined by an enzymatic ‘bridge’ described herein, that may be utilized in various ways in microorganisms, systems and methods of the present invention to biosynthesize butanol and/or isobutanol.

FIGS. 2 and 3 provide calibration curves for butanol and isobutanol obtained using a Coregel Ion310 ion exclusion column.

The paper copy of the sequences provided herein are intended to comply with the basic requirements of applicable Sequence Listing rules, and relevant laws for disclosure of necessary information in a patent application, and may later be supplemented with appropriate electronic or Compact Disk Sequence Listings in a later submission. Descriptions of the sequences are provided in the specification and the appended paper Sequence Listing. The plasmids are derived and modified from native E. coli plasmids.

### DETAILED DESCRIPTION OF EMBODIMENTS OF THE INVENTION

One general aspect of the present invention pertains to microbial biosynthetic pathways for the bio-production of butanol and/or isobutanol from common carbon sources other than petroleum hydrocarbons.

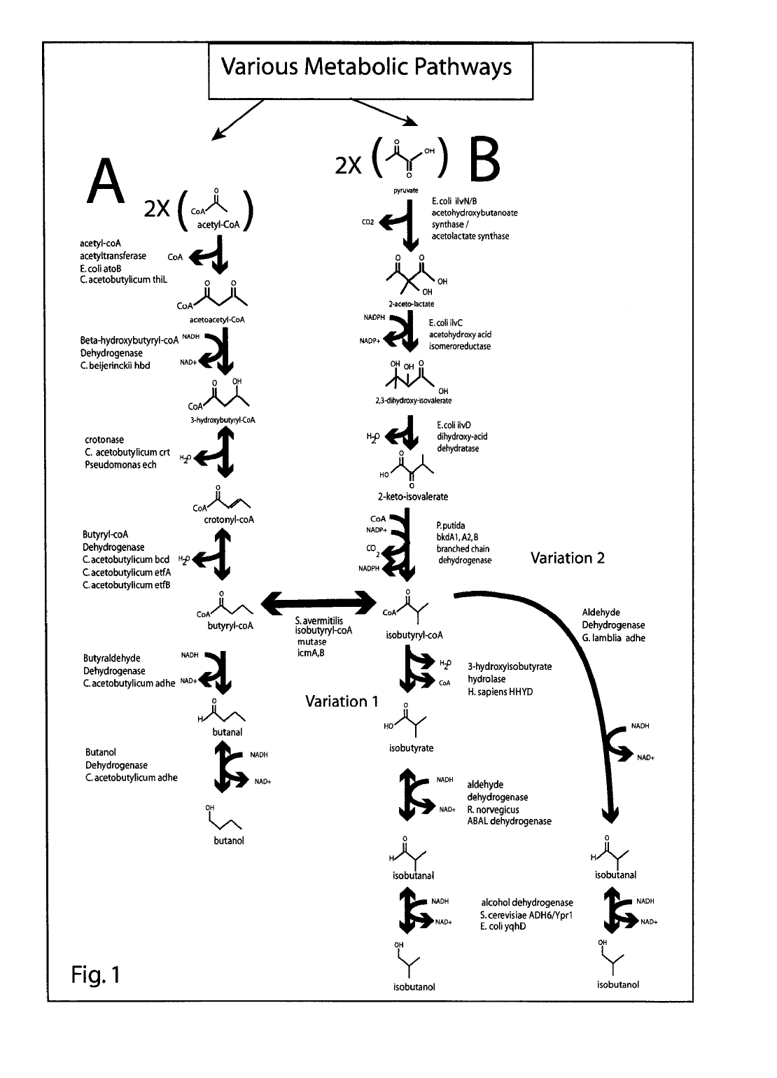

FIG. 1 depicts two pathways in their respective entireties, each showing their respective enzymatic conversion steps, one with two variations for the latter part of that pathway, and also shows the bridge connecting the two pathways. One pathway provides for bio-production of butanol from acetyl-CoA, and the other for bio-production of isobutanol from pyruvate. It is appreciated that when parts of the two pathways are present in a microorganism, and an enzymatic ‘bridge’ as described herein also is present, then a number of alternative pathways may lead to production of butanol and/or isobutanol. These alternative pathways are described below.

Further, it is conceived that by a number of approaches competing metabolic pathways may be modified so that there is less production of undesired metabolic products in a microorganism of the present invention.

Accordingly, the biosynthetic pathways disclosed herein may be utilized in a number of ways to yield, in a particular recombinant microorganism of the present invention, either butanol, isobutanol, or both. As provided herein, genetic modifications may be made to a microorganism of interest not only to provide for these biosynthetic pathways, but also to provide other modifications that, in total, yield a recombinant microorganism that is well-adapted for efficient bio-production of butanol and/or isobutanol in an industrial bio-production system. Various combinations of such genetic modifications, especially the novel combinations of genetic combinations disclosed herein, are believed to advance the art and set the stage for significantly greater economic advantages for industrial bio-production using such recombinant microorganisms. This is perceived to present societal, investment, and corporate opportunities to truly replace or substantially reduce reliance on petroleum hydrocarbons for both industrial chemicals and biofuels. The specific disclosures herein of novel genetic combinations are provided as examples and are by no means intended to limit the scope of combinations contemplated.

As to more detailed aspects of the present invention, the enzyme functions that provide a functional microbial biosynthetic pathway for butanol and/or isobutanol production, and/or other features of the present invention, may be provided in a microorganism of interest by use of a plasmid, or other vector, capable of and adapted to introduce into that microorganism a gene encoding for a respective enzyme having a desired respective function. Mutation and other modifications of genes may also be practiced for various aspects of the invention. Such techniques are widely known and used in the art, and generally may follow methods provided in Sambrook and Russell, Molecular Cloning: A Laboratory Manual, Third Edition 2001 (volumes 1-3), Cold Spring Harbor Laboratory Press, Cold Spring Harbor, N.Y. (“Sambrook and Russell”).

In cases where introduction of more than one gene is required for a particular microorganism, a single vector may be engineered to provide more than one such gene. The two or more genes may be designed to be under the control of a single promoter (i.e., a polycistronic arrangement), or may be under the control of separate promoters and other control regions.

Accordingly, based on the high level of skill in the art and the many molecular biology and related recombinant genetic technologies known to and used by those of skill in the art, there are many approaches to obtaining a recombinant microorganism comprising specific enzymatic properties in particular combinations. The examples provided below are not meant to be limiting of the wide scope of possible approaches to make biological compositions comporting with the present invention, wherein any of those approaches may, without undue experimentation, result in composition(s) that may be used to achieve substantially the same solution as disclosed herein to obtain a desired biosynthetic industrial production of butanol and/or isobutanol.

Referring to FIG. 1, carbohydrates, including sugars, as well as other compounds may be converted to pyruvate and/or acetyl-CoA via well-known metabolic pathways. (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.). Each of the key metabolic intermediates pyruvate and acetyl-CoA may be considered as starting points for specific biosynthetic pathways to butanol and/or isobutanol as discussed in the following paragraphs.

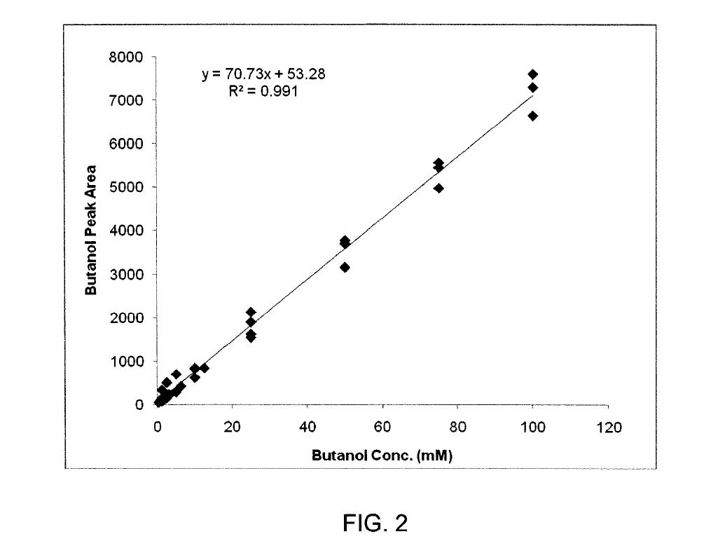

It is noted that natural pathways for production of butanol, isobutanol, and other simple alcohols have been known for well over one decade, if not for many decades (see Functional Genetics of Industrial Yeasts, J. H. de Winde, Ed., Springer-Verlag, Berlin, 2003, incorporated by reference for FIG. 2, page 153, and the discussion on pages 153-154, particularly regarding isobutyl alcohol production and related transformations there from reported by Watanabe et al. in 1993 and Fukuda et al. in 1998; and Color Atlas and Textbook of Diagnostic Microbiology, 5^{th} Ed., E. W. Koneman et al., Lippincott Williams & Wilkins, Philadelphia USA, 1997, incorporated by reference for FIG. 1-17, page 25, showing production of butanol from pyruvate during anaerobic fermentation). More recently, several patent applications have also related to genetic modifications of pathways directed to production of butanol and isobutanol. These include WO2007/041269 A2 and US2007/0092957 A1, which are incorporated by reference for their discussion of the respective pathways.

Considering the existence and knowledge of various naturally occurring biosynthetic pathways, the advances of the present invention are viewed to be founded in some aspects upon the biosynthetic pathways described herein and particular enzymes that may be introduced for them, and also, in further aspects, to other genetic modifications that may be introduced to a recombinant microorganism of this invention, where the latter provide additional benefits for industrial bio-production methods and systems.

A first biosynthetic pathway, identified as biosynthetic pathway A in FIG. 1, may be considered to begin with the enzymatic condensation of two acetyl-CoA molecules to acetoacetyl-CoA. This enzymatic conversion may be done by acetyl-CoA acetyltransferase, such as found in E. coli (atoB) and C. acetobutylicum (thiL). As shown in FIG. 1, and further as known to those skilled in the art, acetyl CoA may be supplied by one or more of a number of metabolic conversions derived from a number of major (and minor) pathways other than the pathways shown in FIG. 1.

Acetoacetyl-CoA is converted to 3-hydroxybutyryl-CoA such as by reaction catalyzed by a β-hydroxybutyryl-CoA dehyrogenase from C. acetobutylicum (hbd) or from C. beijerinckii (hbd). 3-hydroxybutyryl-CoA is converted to crotonyl-CoA such as by the crotonase of C. acetobutylicum (crt) or of Pseudomonas putida (ech). Crotonyl-CoA is converted to butyryl-CoA, such as by one of the butyryl-CoA dehydrogenase enzymes of C. acetobutylicum (bcd, etfA or etfB). The latter reaction is the end of what is considered herein to be the first part of biosynthetic pathway A.

Continuing to the second part of biosynthetic pathway A, butyryl-CoA is converted to butanal, such as by the butyraldehyde dehydrogenase of C. acetobutylicum (adhe). The same enzyme then catalyzes the final step, converting butanal to butanol.

A second biosynthetic pathway, identified as biosynthetic pathway B in FIG. 1, may be considered to begin with the condensation of two pyruvate molecules to 2-aceto-lactate. This may be catalyzed by acetolactate synthase (ilvB and ilvN, a bifunctional enzyme having other catalytic functions), or by other enzymes having equivalent function (for example, acetolactate synthase from Bacillus (alsS) or Klebsiella (bud B) as used in US2007/0092957 A1). As noted in the last-cited reference, substrate specificity is of concern. To improve flux along the desired pathway and reduce or eliminate bio-production of side and/or undesired metabolites and products, appropriate enzyme selection and/or modification may be required. Part of this approach may comprise consideration of alternative enzymes and comparative testing of a selected number of candidate enzymes. This applies to all enzymes (and corresponding nucleic acid sequences) discussed herein.

2-aceto-lactate is converted to 2,3-dihydroxy-isovalerate, such as by the acetohydroxy acid isomeroreductase of E. coli (ilvC). 2,3-dihydroxy-isovalerate is converted to 2-keto-isovalerate such as by the dihydroxy acid dehydratase of E. coli (ilvD). The 2,3-dihydroxy-isovalerate is then converted to isobutyryl-CoA, such as by the branched chain dehydrogenase of P. putida (bkdA1, A2 and B). For each 2,3-dihydroxy-isovalerate molecule this reaction requires one molecule each of coenzyme A (“CoA”) and NADP⁺ and releases one molecule of CO₂. The latter reaction is the end of what is considered herein to be the first part of biosynthetic pathway B.

Continuing to the second part of biosynthetic pathway B, in a first variation isobutyryl-CoA is converted to isobutyrate, releasing CoA and a water molecule, such as by a β-hydroxyisobutyryl-CoA hydrolase from humans (HHYD). Isobutyrate is converted to isobutanal, by an isobutyraldehyde dehydrogenase, such as that conferred by the γ-aminobutyraldehyde dehydrogenase of R. norvegicus (ABAL dehydrogenase) (Testore, G., Colombatto, S., Silvagno, F., and Bedino, S., Purification and kinetic characterization of γ-aminobutyraldehyde dehydrogenase from rat liver, The International Journal of Biochemistry & Cell Biology Volume 27, Issue 11, November 1995, Pages 1201-1210). Finally, isobutanal is converted to isobutanol by any one of a number of candidate alcohol dehydrogenases. For example, either of ADH6 or Ypr1 from S. cerevesiae or yqhD from E. coli may be utilized by introduction into a desired microorganism (or may be used in S. cerevesiae).

A second variation to the second part of biosynthetic pathway B may be utilized in various alternative approaches as described herein. Here isobutyryl-CoA may be converted to isobutyraldehyde, such as by an acylating aldehyde dehydrogenase such as the aldehyde dehydrogenase from G. lamblia adhE. (Sánchez, L. B., Aldehyde Dehydrogenase (CoA-Acetylating) and the Mechanism of Ethanol Formation in the Amitochondriate Protist, Giardia lamblia, Archives of Biochemistry and Biophysics

Volume 354, Issue 1, 1 June 1998, Pages 57-64) Then isobutyraldehyde is converted enzymatically to isobutanol, using any of a group of enzymes generally classified as branched chain alcohol dehydrogenases.

Biosynthetic pathways A and B may be linked by a ‘crossover enzymatic bridge’ so that actyl-CoA may ultimately yield isobutanol, and/or so that pyruvate via 2-aceto-lactate may ultimately yield butanol. This bridge may be accomplished by genetic introduction of a nucleic acid sequence encoding an isobutyryl-CoA mutase enzyme (or its function), such as from S. avermitilis (icma and icmb subunits). The use of an isobutyryl-CoA mutase from a Streptomycete was reported in US2007/0092957 A1 for bridging from acetyl-CoA to an isobutanol pathway. A variation of this approach apparently includes a direct conversion to isobutyraldehyde rather than via isobutyrate. The latter variation is more definitively described herein as the second variation of the second part of biosynthetic pathway B.

Having so described the basic components of two pathways and an isomerase bridge that may connect these pathways, various alternative approaches to practicing the present invention for improved bio-production of butanol and/or isobutanol are discussed.

In a first alternative approach, production of butanol proceeds along biosynthetic pathway A from acetyl-CoA. No genetic modifications are made to enable or enhance biosynthetic pathway B, or genetic modifications are made to reduce or eliminate its production of isobutanol (depending on the microorganism and previous genetic modifications made to it).

In a second alternative approach, production of isobutanol proceeds along biosynthetic pathway B from pyruvate, using the first variation for the second part of biosynthetic pathway B. No genetic modifications may be made to enable or enhance biosynthetic pathway A, or genetic modifications may be made to reduce or eliminate its production of butanol (depending on the microorganism and previous genetic modifications made to it).

In a third alternative approach, production of isobutanol proceeds along biosynthetic pathway B from pyruvate, using the second variation for the second part of biosynthetic pathway B. No genetic modifications may be made to enable or enhance biosynthetic pathway A, or genetic modifications may be made to reduce or eliminate its production of butanol (depending on the microorganism and previous genetic modifications made to it).

In a fourth alternative approach, both biosynthetic pathways A and B (with either of the second part variations of B's second part) are functioning and producing respective quantities of butanol and isobutanol. The crossover enzymatic bridge, such as described above, may or may not be provided.

In a fifth alternative approach, pyruvate is converted successively along the first part of biosynthetic pathway B to yield isobutyryl-CoA. This then is converted to butyryl-CoA by the crossover enzymatic bridge, such as by providing a nucleic acid sequence encoding an isobutyryl-CoA mutase. Then butanol is formed via the bioconversions in the second part of pathway A. The enzymes of the second part of biosynthetic pathway B (either or both variations) are either functional, not provided (depending on the microorganism and genetic modification thereof) or rendered non-functional. If the latter two choices are made, this may result in substantially greater butanol production relative to isobutanol production.

In a sixth alternative approach, acetyl-CoA is converted successively along the first part of biosynthetic pathway A to yield butyryl-CoA. This then is converted to isobutyryl-CoA by the crossover enzymatic bridge, such as by providing a nucleic acid sequence encoding an isobutyryl-CoA mutase. Then isobutanol is formed via the bioconversions in the second part of pathway B, using the first variation disclosed herein. The enzymes of the second part of biosynthetic pathway A are either functional, not provided (depending on the microorganism and genetic modification thereof) or rendered non-functional. If the latter two choices are made, this may result in substantially greater isobutanol production relative to butanol production.

In a seventh alternative approach, acetyl-CoA is converted successively along the first part of biosynthetic pathway A to yield butyryl-CoA. This then is converted to isobutyryl-CoA by the crossover enzymatic bridge, such as by providing a nucleic acid sequence encoding an isobutyryl-CoA mutase. Then isobutanol is formed via the bioconversions in the second part of pathway B, using the second variation disclosed herein. The enzymes of the second part of biosynthetic pathway A are either functional, not provided (depending on the microorganism and genetic modification thereof) or rendered non-functional. If the latter two choices are made, this may result in substantially greater isobutanol production relative to butanol production.

In variations of any such alternative approaches, targeted genetic modifications, mutations or mutated strains may be employed so as to reduce or eliminate production of certain metabolic intermediates and/or end products, the production of which would otherwise lessen the yield of butanol and/or isobutanol from a carbon source in a bio-production event. For example, in one particular embodiment, such as in the mutant strain NZN111 described below (but not limited to that strain), the functioning of D-lactate dehydrogenase (IdhA) is impaired so as to reduce or eliminate the interconversion of pyruvate and lactate. Also, pyruvate formase-lyase (pflB) is impaired so as to reduce or eliminate the conversion of pyruvate to acetate so that substantially less or no acetyl-CoA is formed from pyruvate. These mutations dramatically reduce growth rate in NZN111, however they also present an opportunity, with appropriate further genetic modification, to limit the conversion of carbon sources into undesired byproducts lactate, ethanol and acetate. Use and modification of NZN111 is believed appropriate for alternative approaches that begin with the first part of pathway B, i.e., where a butanol and/or isobutanol pathway includes the conversion of pyruvate to 2-aceto-lactate. Based on the above, these would comprise the second, third, fourth, and fifth alternative approaches.

A second exemplary impairment of an enzyme function involves the use of the strain JW1375, as described below in Examples 17, 19 and 20. This strain comprises an impairment in the functioning of D-lactate dehydrogenase (IdhA) so as to reduce or eliminate the interconversion of pyruvate and lactate. Use and modification of strain JW1375 is believed appropriate for alternative approaches that begin with the first part of pathway A, i.e., where a butanol and/or isobutanol pathway includes the conversion of two acetate molecules to acetoacetyl-CoA. Based on the above, these would comprise the first, fourth, sixth and seventh alternative approaches.

More generally, for any of the alternative approaches genetic modifications may be provided for the reduced production of undesired intermediates or end products of commercial interest, as exemplified above. This may be achieved by various gene deletion and other methods as are known to those skilled in the art in addition to those described herein.

More generally, and depending on the particular metabolic pathways of a microorganism selected for genetic modification, any subgroup of genetic modifications may be made to decrease cellular production of metabolic product(s) selected from the group consisting of acetate, acetoin, acetone, acrylic, malate, fatty acid ethyl esters, isoprenoids, glycerol, ethylene glycol, ethylene, propylene, butylene, isobutylene, ethyl acetate, vinyl acetate, other acetates, 1,4-butanediol, 2,3-butanediol, butanol, isobutanol, sec-butanol, butyrate, isobutyrate, 2-OH-isobutryate, 3-OH-butyrate, ethanol, isopropanol, D-lactate, L-lactate, pyruvate, itaconate, levulinate, glucarate, glutarate, caprolactam, adipic acid, propanol, isopropanol, fusel alcohols, and 1,2-propanediol, 1,3-propanediol, formate, fumaric acid, propionic acid, succinic acid, valeric acid, and maleic acid. Appropriate genetic modification of any one or more of the enzymes that lead to production of these metabolic products decreases or eliminates bio-production of such metabolic product(s). Thus, it is within the scope of the invention to provide one or more genetic modifications effective to decrease or eliminate bio-production of one or more of these metabolic products.

Further, as noted above, the enzymes of the biosynthetic pathways for butanol and isobutanol, and those intended to be modified to reduce production of undesired products and thereby increase butanol and/or isobutanol yield, are exemplary and are not meant to be limiting. The level of skill in biotechnological and genetic recombination arts is high and the knowledge of enzymes is large and ever-expanding, as evidenced by the readily available knowledge that may be found in the art, as exemplified by the information on the following searchable database websites: www.metacyc.org; www.ecocyc.org; and www.brenda-enzymes.info. One skilled in the art is capable with limited research and experimentation to identify any number of genetic sequences either experimentally via directed screening or the assessment of libraries or from sequence databases that encode the desired enzymatic functions. One skilled in the art would then, using the experimental procedures taught in this disclosure, without undue experimentation, be able to express these enzymatic functions in a desired recombinant host.

The enzyme functions to complete a functional microbial biosynthetic pathway for butanol and/or isobutanol production may be provided in a microorganism of interest by use of a plasmid, or other vectors capable of and adapted to introduce into that microorganism a nucleic acid sequence, such as a gene, encoding a polypeptide (including an enzyme) having a desired respective enzymatic function. Other techniques standard in the art allow for the integration of DNA allowing for expression of these enzymatic functions from the genome of numerous microorganisms. These techniques are widely known and used in the art, and generally may follow methods provided in Sambrook and Russell, Molecular Cloning: A Laboratory Manual, Third Edition 2001 (volumes 1-3), Cold Spring Harbor Laboratory Press, Cold Spring Harbor, N.Y.

In cases where introduction of more than one gene is required for a particular microorganism, a single vector may be engineered to provide more than one such gene. The two or more genes may be designed to be under the control of a single promoter (i.e., a polycistronic arrangement), or may be under the control of separate promoters and other control regions. Likewise, nucleic acid sequences encoding polypeptides having two or more respective enzymatic functions (but not comprising the complete amino acid sequence of an enzyme) may be under the control of a single promoter. Thus, to summarize, nucleic acid sequences to encode one or more enzymes (or polypeptides having such enzymatic functions) of any of the above-indicated pathways may be provided to a recombinant microorganism, episomally or integrated into the genome, so as to provide for butanol and/or isobutanol biosynthesis.

Accordingly, based on the high level of skill in the art and the many molecular biology and related recombinant genetic technologies known to and used by those of skill in the art, there are many approaches to obtaining a recombinant microorganism comprising specific enzymatic properties in particular combinations. The examples provided below are not meant to be limiting of the wide scope of possible approaches to make biological compositions comporting with the present invention, wherein any of those approaches may, without undue experimentation, result in composition(s) that may be used to achieve substantially the same solution as disclosed herein to obtain a desired biosynthetic industrial production of butanol and/or isobutanol.

In the following examples, efforts have been made to ensure accuracy with respect to numbers used (e.g., amounts, temperatures, etc.), but some experimental error and deviation should be accounted for. Unless indicated otherwise, temperature is in degrees Celsius and pressure is at or near atmospheric pressure at approximately 5340 feet (1628 meters) above sea level. All reagents, unless otherwise indicated, were obtained commercially.

The meaning of abbreviations is as follows: “C” means Celsius or degrees Celsius, as is clear from its usage, “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “μM” means micromolar, “M” means molar, “mmol” means millimole(s), “μmol” means micromole(s)”, “g” means gram(s), “μg” means microgram(s) and “μg” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v” means volume/volume percent, “IPTG” means isopropyl-μ-D-thiogalactopyranoiside, “RBS” means ribosome binding site, “HPLC” means high performance liquid chromatography, and “GC” means gas chromatography.

### EXAMPLES

The following pertain to exemplary methods of modifying specific species of host organisms that span a broad range of microorganisms of commercial value. As noted elsewhere, these examples are not meant to be limiting of the scope of the present invention.

Where there is a method to achieve a certain result that is commonly practiced in two or more specific examples, that method may be provided in a separate Common Methods section that follows the examples. Each such common method is incorporated by reference into the respective specific example that so refers to it. Also, where supplier information is not complete in a particular example, additional manufacturer information may be found in a separate Summary of Suppliers section that may also include product code, catalog number, or other information. This information is intended to be incorporated in respective specific examples that refer to such supplier and/or product.

### Example 1

### Cloning of S. avermitilis icmA and icmB

A nucleic acid sequence encoding the protein sequence for the isobutyryl-CoA mutase subunits A and B from S. avermitilis was codon optimized for enhanced protein expression in E. coli according to a service from DNA 2.0 (Menlo Park, Calif. USA), a commercial DNA gene synthesis provider. The thus-codon-optimized nucleic acid sequence encoding an operon containing both the icmA and icmB genes incorporated an EcoRI restriction site upstream of the gene open reading frames and was followed by a EcorV restriction site. In addition Shine Delgarno sequences or ribosomal binding sites were placed in front of the respective start codons of each of the two nucleic acid sequences for the subunits A and B of isobutyryl-CoA mutase. This nucleic acid sequence (SEQ ID NO:0001) was synthesized by DNA 2.0 and provided in a pJ206 vector backbone.

### Example 2

### Cloning of C. acetobutylicum adhe Gene

C. acetobutylicum DSMZ # 792/ATCC #824 was obtained from DSMZ and cultures grown as described in Subsection I of the Common Methods Section, below. Genomic DNA from C. acetobutylicum cultures was obtained from a Qiagen genomic DNAEasy kit according to manufacturer's instructions. The following oligonucleotides were obtained from the commercial provider Operon. Primer 1: CTCTCCCGGGTATAAGGCATCAAAGTGTGT (SEQ ID NO:0026) and Primer 2: CTCTCCCGGGCTCGAGGTCTATGTGCTTCATGAAGC (SEQ ID NO:0027). Primer 1 contains a SmaI restriction site and a Shine-Delgarno sequence while Primer 2 contains both a SmaI and a Not I restriction site. These primers were used to amplify the adhe region from C. acetobutylicum genomic DNA using standard polymerase chain reaction (PCR) methodologies. The predicted sequence of the resultant PCR product is given in (Seq ID 0002). The adhe PCR product was ligated into pSC-B-amp/kan (Seq ID:0003) and transformed according to manufacturer's instructions. The predicted sequence of the resultant plasmid is given in (SEQ ID NO:0012).

### Example 3

### Cloning of P. putida bkd A1, A2, B Genes

P. putida strain KT2440 was a gift from the Gill lab (University of Colorado at Boulder) and was obtained as an actively growing culture. Cultures were grown as described in in Subsection I of the Common Methods Section, below. Genomic DNA from P. putida cultures was obtained from a Qiagen genomic DNAEasy kit according to manufacturer's instructions. The following oligonucleotides were obtained from the commercial provider Operon. Primer 1: GATCGAATTCAATTGAAAAAGGAAGAGTATGAACGAGTACGCGCCCCTTGCG (SEQ ID NO:0028) and Primer 2: GATCAAGCTTCGCCGATGATCAACAGGGTTGTC (SEQ ID NO:0029). Primer 1 contains a EcoRI restriction site and a Shine-Delgarno sequence while Primer 2 contains a Hind III restriction site. These primers were used to amplify the bkd A1, A2, B region from P. putida genomic DNA using standard polymerase chain reaction (PCR) methodologies. The predicted sequence of the resultant PCR product is given in (SEQ ID NO:0008). The bkd A1, A2, B PCR product was ligated into pSC-B-amp/kan (SEQ ID NO:0003) and transformed according to manufacturer's instructions. The predicted sequence of the resultant plasmid is given in (SEQ ID NO:0013).

### Example 4

### Cloning of C. acetobutylicum thiL (Prophetic)

C. acetobutylicum DSMZ # 792/ATCC #824 is obtained from DSMZ and cultures are grown as described in Subsection I of the Common Methods Section, below. Genomic DNA from C. acetobutylicum cultures is obtained from a Qiagen genomic DNAEasy kit according to manufacturer's instructions. The following oligonucleotides are obtained from the commercial provider Operon. Primer 1: ATCCCGGGGAGGAGTAAAACATGAGAGA (SEQ ID NO:0030) and Primer 2: ATCCCGGGCTCGAGTTAGTCTCTTTCAACTACGA (SEQ ID NO:0031). Primer 1 contains a SmaI restriction site while Primer 2 contains both a SmaI and a XhoI restriction site. These primers are reported to be used to amplify the thiL region from C. acetobutylicum genomic DNA using standard polymerase chain reaction (PCR) methodologies (Inui et al, Applied Genetics and Molecular Biotechnology. (2008), 77:1305-1316). The predicted sequence of the resultant PCR product is given in (SEQ ID NO:0004). This sequence is subclonable into any number of commercial cloning vectors including but not limited to pCR2.1-topo (Invitrogen), other topo-isomerase based cloning vectors (Invitrogen) the pSMART-series of cloning vectors from Lucigen or the Strataclone series of vectors. (Stratagene) after amplification by PCR.

### Example 5

### Cloning of C. acetobutylicum crt, bcd, etfB, etfA and hbd Genes (Prophetic)

C. acetobutylicum DSMZ # 792/ATCC #824 is obtained from DSMZ and cultures are grown as described in Subsection I of the Common Methods Section, below. Genomic DNA from C. acetobutylicum cultures is obtained from a Qiagen genomic DNAEasy kit according to manufacturer's instructions. The following oligonucleotides are obtained from the commercial provider Operon. Primer 1: ATCCCGGGATATTTTAGGAGGATTAGTCATGGAACTAAACAATG (SEQ ID NO:0032) and Primer 2: ATCCCGGGAGATCTTGTAAACTTA TTTTGAATAA TCGTAGAAACCC (SEQ ID NO:0033). Primer 1 contains a SmaI restriction site while Primer 2 contains both a SmaI and a BglII restriction site. These primers are used to amplify the crt, bcd, etfB, etfA, hbd operon region from C. acetobutylicum genomic DNA using standard polymerase chain reaction (PCR) methodologies. The predicted sequence of the resultant PCR product is given in (SEQ ID NO:0005). This sequence is subclonable into any number of commercial cloning vectors including but not limited to pCR2.1-topo (Invitrogen), other topo-isomerase based cloning vectors (Invitrogen) the pSMART-series of cloning vectors from Lucigen or the Strataclone series of vectors (Stratagene) after amplification by PCR.

### Example 6

### Cloning of E. coli ilv N/B Gene

E. Coli K12 CGSC # 4401 was obtained as a kind gift from the laboratory of Prof. Ryan T. Gill from the University of Colorado at Boulder and cultures grown as described in Subsection I of the Common Methods Section, below. Genomic DNA from E. coli cultures was obtained from a Qiagen genomic DNAEasy kit according to manufacturer's instructions. The following oligonucleotides were obtained from the commercial provider Operon. Primer 1: GATCGAATTCAAAGTCGGCC CAGAAGAAAA GGACTGGAGC ATGGCAAGTT CGGGCACAAC (SEQ ID NO:0034) and Primer 2: GATCCTCGAGTGTCCTGGCG GGTAAAAAAA ATACGCGCTT ACCTTAACGA TAAGCGCGAT GTTGTTCAAG (SEQ ID NO:0035). Primer 1 contains a EcoRI restriction site and a Shine-Delgarno sequence while Primer 2 contains a XhoI restriction site. These primers were used to amplify the ilv N/B region from E. coli genomic DNA using standard polymerase chain reaction (PCR) methodologies. The predicted sequence of the resultant PCR product is given in (SEQ ID NO:0006). The Ilv N/B PCR product was cloned into pCR2.1 TOPO-TA (SEQ ID NO:0014) and transformed according to manufacturer's instructions. The predicted sequence of the resultant plasmid is given in (SEQ ID NO:0007).

### Example 7

### Cloning of E. coli ilv C Gene

E. coli K12 CGSC # 4401 was obtained as a kind gift from the laboratory of Prof. Ryan T. Gill from the University of Colorado at Boulder. Cultures of this were grown as described in Subsection I of the Common Methods Section, below. Genomic DNA from E. coli cultures was obtained from a Qiagen genomic DNAEasy kit according to manufacturer's instructions. The following oligonucleotides were obtained from the commercial provider Operon. Primer 1: GATCGTCGACATAAGAAGCA CAACATCACG AGGAATCACC ATGGCTAACT ACTTCAATAC (SEQ ID NO:0036) and Primer 2: GATCTCTAGACAGCGCGCAC TTAACCCGCA ACAGCAATAC GTTTCATATC TGTCATATAG (SEQ ID NO:0037). Primer 1 contains a Sal I restriction site and a Shine-Delgarno sequence while Primer 2 contains an Xba I restriction site. These primers were used to amplify the ilv C region from E. coli genomic DNA using standard polymerase chain reaction (PCR) methodologies. The predicted sequence of the resultant PCR product is given in (SEQ ID NO:0015). The Ilv C PCR product was cloned into pCR2.1 topo-TA (SEQ ID NO:0014) and transformed according to manufacturer's instructions. The predicted sequence of the resultant plasmid is given in (SEQ ID NO:0016).

### Example 8

### Cloning of E. coli ilv D Gene

E. Coli K12 CGSC # 4401 was obtained as a kind gift from the laboratory of Prof. Ryan T. Gill from the University of Colorado at Boulder and cultures grown as described in Subsection I of the Common Methods Section, below. Genomic DNA from E. coli cultures was obtained from a Qiagen genomic DNAEasy kit according to manufacturer's instructions. The following oligonucleotides were obtained from the commercial provider Operon. Primer 1: GATCTCTAGACCGTCCCATT TACGAGACAG ACACTGGGAG TAAATAAAGT (SEQ ID NO:0038) and Primer 2: GATCGCGGCC GCGGGTTGCG AGTCAGCCAT TATTAACCCC CCAGTTTCGA TT (SEQ ID NO:0039). Primer 1 contains an Xba I restriction site and a Shine-Delgarno sequence while Primer 2 contains a Not I restriction site. These primers were used to amplify the ilv D. The predicted sequence of the resultant PCR product is given in (SEQ ID NO:0017). The Ilv D PCR product was cloned into Topo 2.1 (SEQ ID NO:0014) and transformed according to manufacturer's instructions. The predicted sequence of the resultant plasmid is given in (SEQ ID NO:0018).

### Example 9

### Construction of Cloning Vector pKK223-MCS1

A circular plasmid based cloning vector termed pKK223-MCS1 for expression of genes for butanol and/or isobutanol syntheses in E. coli was constructed as follows. An E. coli cloning strain bearing pKK223-aroH was obtained as a kind gift from the laboratory of Prof. Ryan T. Gill from the University of Colorado at Boulder. Cultures of this strain bearing the plasmid were grown by standard methodologies and plasmid DNA was prepared by a commercial miniprep column from Qiagen. Plasmid DNA was digested with the restriction endonucleases EcoR I and HindIII obtained from New England BioLabs according to manufacturer's instructions. This digestion served to separate the aroH reading frame from the pKK223 backbone. The digestion mixture was separated by agarose gel electrophoresis, and visualized under UV transillumination as described Subsection II of the Common Methods Section, below. An agarose gel slice containing a DNA piece corresponding to the backbone of the pKK223 plasmid was cut from the gel and the DNA recovered with a standard gel extraction protocol and components from Qiagen according to manufacturer's instructions. The following oligonucleotides were obtained from the commercial provider Operon. Oligo 1: [Phos]AATTCGCAT TAAGCTTGCA CTCGAGCGTC GACCGTTCTA GACGCGATATCCGAATCCCG GGCTTCGTGC GGCCGC (SEQ ID NO:0040) and Oligo 2: [Phos]AGCTGCGGCC GCACGAAGCC CGGGATTCGG ATATCGCGTC TAGAACGGTC GACGCTCGAG TGCAAGCTTA ATGCG (SEQ ID NO:0041). [Phos] indicates a 5′ phosphate. These oligonucleotides were mixed in a 1:1 ratio 50 micromolar concentration in a volume of 50 microliters and hybridized to a double stranded piece of DNA in a thermocycler with the following temperature cycles—95 C for 10 minutes, 90 C for 5 minutes, 85 C for 10 minutes, 80 C for 5 minutes, 75 C for 5 minutes, 70 C for 1 minutes, 65 C for 1 minutes, 55 C for 1 minutes, and then cooled to 4 C. This double stranded piece of DNA has 5 overhangs corresponding to overhangs of EcoR I and Hind III restriction sites. This piece was diluted in Deionized water 1:100 and ligated according to and with components of the Ultraclone Cloning (Lucigen). into the gel extracted EcoR I, Hind III digested pKK223 backbone. The ligation product was transformed and electroporated according to manufacturer's instructions. The sequence of the resulting vector termed pKK223-MCS1 (Seq. ID 0019) was confirmed by routine sequencing performed by the commercial service provided by Macrogen (USA). pKK223-MCS1 confers resistance to beta-lactamase and contains a new multiple cloning site and a ptac promoter inducible in E. coli hosts by IPTG.

### Example 10

### Construction of Cloning Vector pKK223-MCS2

A circular plasmid based cloning vector termed pKK223-MCS2 for expression of genes for butanol and/or isobutanol syntheses in E. coli was constructed as follows. An E. coli 10 G F′ cloning strain (Lucigen, Madison Wis.) bearing pKK223-MCS1 was obtained from example 8. Cultures of this strain bearing the plasmid were grown by standard methodologies and plasmid DNA was prepared by a commercial miniprep column from Qiagen. Plasmid DNA was digested with the restriction endonuclease XbaI and treated with antarctic phosphatase, both enzymes were obtained from New England BioLabs and reactions carried out according to manufacturer's instructions. This digestion served to linearize the vector backbone. The digestion mixture was separated by agarose gel electrophoresis, and visualized under UV transillumination as described in Subsection II of the Common Methods Section, below. An agarose gel slice containing a DNA piece corresponding to the backbone of the linear vector was cut from the gel and the DNA recovered with a standard gel extraction protocol and components from Qiagen according to manufacturer's instructions. The following oligonucleotides were obtained from the commercial provider Operon. Oligo 1: CTAG TTTAAA CATATTCTGA AATGAGCTGT TGACAATTAA TCATCGGCTC GTATAATGTG (SEQ ID NO:0042), Oligo 2: [Phos] TGGAATTGTG AGCGGATAAC AATTTCACAC ACAT (SEQ ID NO:0043), Oligo 3: CTAGATGTGTGTGAAATTGT TATCCGCTCA CAATTCCACA CATTATACGAGCCGATGA (SEQ ID NO:0044) and Oligo 4: [Phos] TTAATTGTCA ACAGCTCATT TCAGAATATG TTTAAA (SEQ ID NO:0045). [Phos] indicates a 5′ phosphate. These oligonucleotides were mixed in a 1:1 ratio 50 micromolar concentration in a volume of 50 microliters and hybridized to a double stranded piece of DNA in a thermocycler with the following temperature cycles. 95 C for 10 minutes, 90 C for 5 minutes, 85 C for 10 minutes, 80 C for 5 minutes, 75 C for 5 minutes, 70 C for 5 minutes, 65 C for 5 minutes, 60 C for 5 minutes, 55 C for 10 minutes, 50 C for 10 minutes, 45 C for 5 minutes, 40 C for 5 minutes, and then cooled to 4 C. This double stranded piece of DNA has 5 overhangs corresponding to overhangs of an XbaI restriction sites. This piece is diluted in Deionized water 1:100 and ligated according to and with components of the Ultraclone Cloning (Lucigen) into the gel extracted XbaI digested and antarctic phosphatase treated pKK223-MCS1. The ligation product is transformed and electroporated according to manufacturer's instructions. The predicted sequence of the resulting vector termed pKK223-MCS1 (Seq. ID 0010) is confirmed by routine sequencing performed by the commercial service provided by Macrogen (USA). pKK223-MCS2 confers resistance to beta-lactamase and contains 2 ptac promoters inducible in E. coli hosts by IPTG associated with 2 multiple cloning sites.

### Example 11

### Construction of Cloning Vector pACYC177-MCS1 (Prophetic)

A circular plasmid based cloning vector termed pACYC177-MCS1 for expression of nucleic acid sequences involved in isobutanol and butanol synthesis in E. coli is constructed as follows. An E. coli cloning strain bearing pKK223-aroH is obtained as a kind gift from the laboratory of Prof. Ryan T. Gill from the University of Colorado at Boulder. Plasmid pACYC177 is obtained from the commercial provider New England Biolabs. These two plasmids are propagated by standard methodologies and plasmid DNA is prepared by a commercial miniprep columns from Qiagen. The following oligonucleotides are obtained from the commercial provider Operon. Primer 1: GAGCGTCAGACCCC (SEQ ID NO:0046) Primer2: GTCAAGTCAGCGTAATGC (SEQ ID NO:0047) Primer 3: [phos]TGCACCAATGCTTCTGG (SEQ ID NO:0048) Primer 4: [phos]GAAAAATAAACAAAAGAGTTTGTAGAAACGC (SEQ ID NO:0049). [Phos] indicates a 5′ phosphate, and thus the 5′ end. Primers 1 and 2 are used to amplify the vector backbone of pACYC177 including the kanamycin resistance gene and origin of replication by standard polymerase chain reaction methods. Primers 2 and 3 are used to amplify the ptac promoter aroH gene and rrnB terminator from pKK223-aroH by standard polymerase chain reaction methods. The two separate PCR products are individually separated by agarose gel electrophoresis, and are visualized under UV transillumination as described in the Common Methods Section, subsection II. Agarose gel slices containing the appropriate DNA pieces are cut from the gel and the DNA is recovered with a standard gel extraction protocol and components from Qiagen according to manufacturer's instructions. After gel purification the two PCR products are ligated together and are electroporated into an E. coli cloning host yielding the plasmid pACYC177-ptac-aroH. pACYC177-ptac-aroH plasmid DNA is digested with the restriction endonucleases EcorI and HindIII obtained from New England BioLabs according to manufacturer's instructions. This digestion serves to separate the aroH reading frame from the pACYC177-ptac backbone. The digestion mixture is separated by agarose gel electrophoresis, and is visualized under UV transillumination as described in the Common Methods Section, subsection II. An agarose gel slice containing a DNA piece corresponding to the backbone of the pACYC177-ptac plasmid is cut from the gel and the DNA is recovered with a standard gel extraction protocol and components from Qiagen according to manufacturer's instructions.

The following oligonucleotides are obtained from the commercial provider Operon. Oligo 1: [Phos]AATTCGCAT TAAGCTTGCA CTCGAGCGTC GACCGTTCTA GACGCGATATCCGAATCCCG GGCTTCGTGC GGCCGC (SEQ ID NO:0050) and Oligo 2: [Phos]AGCTGCGGCC GCACGAAGCC CGGGATTCGG ATATCGCGTC TAGAACGGTC GACGCTCGAG TGCAAGCTTA ATGCG (SEQ ID NO:0022). [Phos] indicates a 5′ phosphate. These oligonucleotides are mixed in a 1:1 ratio 50 micromolar concentration in a volume of 50 microliters and are hybridized to form by annealing a double stranded piece of DNA in a thermocycler with the following temperature cycles. 95 C for 10 minutes, 90 C for 5 minutes, 85 C for 10 minutes, 80 C for 5 minutes, 75 C for 5 minutes, 70 C for 1 minutes, 65 C for 1 minutes, 55 C for 1 minutes, and then cool to 4 C. The resultant double stranded piece of DNA has 5′ overhangs corresponding to overhangs of EcorI and HindIII restriction sites. This piece of DNA, which comprises multiple cloning sites, is diluted in Deionized water 1:100 and is ligated according to and with components of the Ultraclone Cloning Kit (Lucigen) into the gel extracted EcorI, HindIII digested pACYC177-ptac backbone. The ligation product is transformed and electroporated according to manufacturer's instructions. The predicted sequence of the resulting vector termed pACYC177-MCS1 (SeqID 0011) is confirmed by routine sequencing performed by the commercial service provided by Macrogen (USA). pACYC177-MCS1 confers resistance to beta-lactamase and contains a new multiple cloning site and a ptac promoter inducible in E. coli hosts by IPTG.

### Example 12

### Subcloning bkd into pKK223-MCS2 (Prophetic)

Cultures of strains bearing the pSC-B-amp/kan-bkda1, a2, b and the pKK223-MCS2 plasmids are grown by standard methodologies and plasmid DNA is prepared by a commercial miniprep column from Qiagen. Plasmid pSC-B-amp/kan-bkda1,a2,b DNA is digested with the restriction endonucleases EcoRI I and Hind III to obtained from New England BioLabs according to manufacturer's instructions. This digestion serves to separate the bkdA1, A2, B reading frames from the pSC-B-amp/kan backbone. The digestion mixture is separated by agarose gel electrophoresis, and is visualized under UV transillumination as described in Subsection II of the Common Methods Section, below. Plasmid pKK223-MCS2 DNA also is digested with the restriction endonucleases EcoRI I and Hind III obtained from New England BioLabs according to manufacturer's instructions. An agarose gel slice containing a DNA piece corresponding to pKK223-MCS2 is cut from the gel and the DNA is recovered with a standard gel extraction protocol and components from Qiagen according to manufacturer's instructions. An agarose gel slice containing a DNA piece corresponding to the backbone of the pKK223-MCS2 plasmid is cut from the gel and the DNA is recovered with a standard gel extraction protocol and components from Qiagen according to manufacturer's instructions. The DNA fragment bkd A1, A2, B is ligated into the cut pKK223-MCS2 and transformed following standard molecular biology protocols. The predicted sequence of the resultant plasmid is given in (SEQ ID NO:0020).

### Example 13

### Subcloning adhe into pKK223-MCS2-bkd A1,A2,B (Prophetic)

Cultures of strains bearing the pSC-B-amp/kan-adhe and the pKK223-MCS2-bkd A1, A2, plasmids are grown by standard methodologies and plasmid DNA is prepared by a commercial miniprep column from Qiagen. Plasmid pSC-B-amp/kan-adhe DNA is digested with the restriction endonucleases Sam I and Not I obtained from New England BioLabs according to manufacturer's instructions. This digestion serves to separate the adhe reading frames from the pSC-B-amp/kan backbone. The digestion mixture is separated by agarose gel electrophoresis, and is visualized under UV transillumination as described in Subsection II of the Common Methods Section, below. Plasmid pKK223-MCS2-bkd A1, A2, DNA also is digested with the restriction endonucleases SmaI and Not I obtained from New England BioLabs according to manufacturer's instructions. An agarose gel slice containing a DNA piece corresponding to pKK223-MCS2-bkd A1, A2, is cut from the gel and the DNA is recovered with a standard gel extraction protocol and components from Qiagen according to manufacturer's instructions. The DNA fragment adhe is ligated into the cut pKK223-MCS2-bkd A1, A2, and is transformed following standard molecular biology protocols. The predicted sequence of the resultant plasmid pKK223-MCS2-bkd A1, A2-adhe is given in (SEQ ID NO:0021).

### Example 14

### Subcloning icm A,B into pKK223-MCS2-bkd A1,A2β,B-adhe (Prophetic)

Cultures of strains bearing the pJ206-icm A, B and the pKK223-MCS2-bkd A1, A2, B-adhe, plasmids will be grown by standard methodologies and plasmid DNA will be prepared by a commercial miniprep column from Qiagen. Plasmid pJ206-icm A,B DNA will be digested with the restriction endonucleases NheI and EcoRv I obtained from New England BioLabs according to manufacturer's instructions. This digestion will serve to separate the icm A, B reading frames from the pJ206 backbone. The digestion mixture will be separated by agarose gel electrophoresis, and visualized under UV transillumination as described in Subsection II of the Common Methods Section, below. Plasmid pKK223-MCS2-bkd A1, A2, B-adhe DNA will also be digested with the restriction endonucleases Xba I (which has a compatible sticky end to NheI I) and SmaI obtained from New England BioLabs according to manufacturer's instructions. An agarose gel slice containing a DNA piece corresponding to plasmid pKK223-MCS2-bkd A1, A2, B-adhe, will be cut from the gel and the DNA recovered with a standard gel extraction protocol and components from Qiagen according to manufacturer's instructions. The DNA fragment icm A,B is ligated into the cut pKK223-MCS2-bkd A1,A2,B-adhe and is transformed following standard molecular biology protocols. The predicted sequence of the resultant plasmid OPXpBut1 is given in (SEQ ID NO:0023).

### Example 15

### Cloning of Human β-Hydroxyisobutyrl-Coenzyme A Hydrolase (Prophetic)

The protein sequence for the β-Hydroxyisobutyrl-coenzyme-A hydrolase from H. sapiens will be codon optimized for E. coli according to a service from DNA 2.0 a commercial DNA gene synthesis provider. The DNA sequence encoding the gene will be synthesized with proper 5-prime (“5′”) and 3-prime (“3′”) restriction sites for sub-cloning into expression cassettes as well as a Shine-Delgarno sequences or ribosomal binding site will be placed in front of the start codon of the gene. The predicted nucleic acid sequence construct (Seq ID: 0024) will synthesized by DNA 2.0 and provided in a commercially available vector backbone, such as but not limited to those described in this application.

### Example 16

### Subcloning ilv N/B, ilv C, IlvD into Expression Cassette pACYC-MCS1 (Prophetic)

To increase flux from pyruvate to 2-keto-isovalerate, ilv N/B, ilv C, IlvD will be subcloned into the expression cassette pACYC-MCS1 using standard molecular biology protocols similar to those discussed in examples 9, 10, 11 and 12.

### Example 17

### OPXpbut1 and pACYC-MCS1-ilv N/B, ilv C, IlvD will be Coexpressed in the E. coli Strain NZNIII (Prophetic)

Co-expression of OPXpbut1 and pACYC-MCS1-ilv N/B, ilv C, IlvD in NZNIII will lead to the formation of butanol from pyruvate as outlined in FIG. 1. Further, the NZN111 strain of E. coli comprises a functional defect in idhA and pflB. idhA encodes the enzyme lactate dehydrogenase, so that production of lactate from pyruvate is substantially reduced or eliminated, and pflB encodes a pyruvate formate-lyase so that production of formate and acetyl-CoA from pyruvate is substantially reduced or eliminated. This results in lower production of undesired products and accordingly in increased percentage yield of butanol, such as in a bio-production event. Optimal growth and induction protocols will be determined following standard molecular biology protocols and butanol production will be determined by HPLC as outlined in general methods.

### Example 18

### Subcloning C. acetobutylicum crt, bcd, etfB, etfA, hbd, thiL and adhe into Expression Cassette pK223-MCS2 and Transformation into JW1375 Idha⁻ for Butanol Production (Prophetic)

Expression of C. acetobutylicum genes crt, bcd, etfB, etfA, hbd, thiL and adhe in E. coli are reported to convert acetyl-CoA to butanol (M. Inui et al., Expression of Clostridium acetobutylicum butanol synthetic genes in Escherichia coli, Appl Microbiol Biotechnol (2008) 77:1305-1316). C. acetobutylicum genes crt, bcd, etfB, etfA, hbd, thiL and adhe will be subcloned into pkk223-MCS2 using standard molecular biology protocols as outlined in examples 9, 10, 11 and 12. The resulting plasmid will be expressed in E. coli strain JW1375 Idha⁻. This strain comprises a functional defect in IdhA, which encodes the enzyme lactate dehydrogenase, so that production of lactate from pyruvate is substantially reduced or eliminated. This results in lower production of undesired products and accordingly in increased percentage yield of butanol, such as in a bio-production event. Optimal growth and induction protocols will be determined following standard molecular biology protocols and butanol production will be determined by HPLC as outlined in general methods.

### Example 19

### Conversion of Pyruvate to Isobutanol by Co-Expression of pACYC-MCS1-ilv N/B, ilv C, IlvD and pKK223-bkd A1,A2,B-adhe-ADH6 (Prophetic)

Co-expression of the two plasmids named immediately above will convert pyruvate to isobutanol. Construction of pACYC-MCS1-ilv N/B, ilv C, IlvD is described in example 16. The ADH6 gene from S. cerevisiae will be amplified by PCR from genomic DNA with compatible restriction sites and a Shine-Delgarno sequence such that it can be cloned into pKK223-MCS2-bkd A1, A2, B-adhe. This pathway is disclosed in U.S. patent publication number US2007/0092957 A1. This patent publication is here in incorporated by reference particularly for its teachings of the noted pathway section from isobutyrl-CoA to isobutyraldehyde utilizing acylating aldehyde dehydrogenase enzymes such as C. acetobutylicum adhe, adhe1, C. beijerinckii ald, and P. putida nahO. Also noted is the conversion of isobutyraldehyde to isobutanol using E. coli yqhD, S. cerevisiae YPR1 or ADH6.

### Example 20

### Conversion of acetyl-CoA to Isobutanol by Co-Expression of pKK223-MCS2-thiL-crt, bcd, etfB, etfA, hbd-icm A, B and pACYC-MCS1-adhe-adh6 (Prophetic)

This pathway converts acetyl-CoA to isobutanol by utilizing C. acetobutylicum genes crt, bcd, etfB, etfA, hbd, and thiL to convert acetyl-CoA to butyrl-CoA followed by the conversion of butryl-CoA to isobutryl-CoA by isobutryl-coA mutase subunits A and B from S. avermitilis. Isobutyryl-CoA is then converted to isobutanol such as by the approach described in example 29. The resulting plasmids will be expressed in E. coli strain JW1375 Idha⁻. This strain comprises a functional defect in IdhA, which encodes the enzyme lactate dehydrogenase, so that production of lactate from pyruvate is substantially reduced or eliminated. This results in lower production of undesired products and accordingly in increased percentage yield of isobutanol, such as in a bio-production event. Optimal growth and induction protocols will be determined following standard molecular biology protocols and isobutanol production will be determined by HPLC as outlined in general methods.

### Example 21

### Conversion of acetyl-CoA to Isobutanol by co-expression of pKK223-MCS2-thiL-crt, bcd, etfB, etfA, hbd-icm A, B and pACYC-MCS1-HHYD-ABAL Dehydrogenase-ADH6 (Prophetic)

This pathway can be utilized to convert acetyl-CoA to isobutanol. The pathway from acetyl-CoA to isobutyl-CoA is the same as described in example 18. Isobutyl-CoA will then be converted to isobutyrate by Human 6-Hydroxyisobutyryl-coenzyme A hydrolase (HHYD). This enzyme has been isolated and shown to have activity for isobutyryl-CoA. (Hawes et. al., The Journal of Biological Chemistry Vol. 271, No. 42 pp. 26430-26434, 1996). HHYD enzyme activity could also be optimized using standard metabolic engineering techniques to increase isobutanol production. Isobutyrate will then be converted to isobutanal by an aldehyde dehyrogenase such as the γ-aminobutyraldehyde dehydrogenase of R. norvegicus (ABAL dehydrogenase). Isobutanal is then converted to isobutanol by the alcohol dehydrogenase ADH6 as described in example 19. The resulting plasmids encoding the pathway described above will be expressed in E. coli strain JW1375 Idha⁻. This strain comprises a functional defect in IdhA, which encodes the enzyme lactate dehydrogenase, so that production of lactate from pyruvate is substantially reduced or eliminated. This results in lower production of undesired products and accordingly in increased percentage yield of isobutanol, such as in a bio-production event. Optimal growth and induction protocols will be determined following standard molecular biology protocols and isobutanol production will be determined by HPLC as outlined in general methods.

### Example 22

### Conversion of Pyruvate to Isobutanol by Co-Expression of pACYC-MCS1-ilv N/B, ilv C, IlvD and pKK223-bkd A1,A2,B-HHYD-ABAL-ADH6 (Prophetic)

Coexpression of pACYC-MCS1-ilv N/B, ilv C, IlvD and pKK223-bkd A1, A2, B-HHYD-ABAL dehydrogenase-ADH6 will convert pyruvate to isobutanol. Co-expression of pACYC-MCS1-ilv N/B, ilv C, IlvD and pKK223-bkd A1, A2, B-HHYD-ABAL-ADH6 in NZNIII will lead to the formation of isobutanol from pyruvate as outlined in FIG. 1. The NZN111 strain of E. coli comprises a functional defect in idhA and pfIB. idhA encodes the enzyme lactate dehydrogenase, so that production of lactate from pyruvate is substantially reduced or eliminated, and pflB encodes a pyruvate formate-lyase so that production of formate and acetyl-CoA from pyruvate is substantially reduced or eliminated. This results in lower production of undesired products and accordingly in increased percentage yield of isobutanol, such as in a bio-production event. Optimal growth and induction protocols will be determined following standard molecular biology protocols and isobutanol production will be determined by HPLC as outlined in general methods.

All restriction endonucleases and Antarctic phosphatase obtained from New England BioLabs and all reactions carried out according to manufacturer's instructions. Cultures of an E. coli cloning strains bearing subclones are cultured according to standard methodologies and all plasmid DNA prepared by a commercial miniprep column from Qiagen. The digestion mixtures are separated by routine agarose gel electrophoresis, and visualized under UV transillumination as described in Subsection II of the Common Methods Section, below. Agarose gel slices containing desired DNA pieces are cut from the gel and the DNA recovered with a standard gel extraction protocol and components from Qiagen according to manufacturer's instructions. Ligations and transformations are also carried out as described in Subsection II of the Common Methods Section, below.

### Common Methods Section

All methods in this Section are provided for incorporation into the above methods where so referenced therein. When incorporated into an actual example (in contrast to a prophetic example), the indicated steps actually occurred.

Subsection I. Bacterial Growth Methods: Bacterial growth culture methods, and associated materials and conditions, are disclosed for respective species as follows. If any species listed below is not specifically discussed for use in an example above, nonetheless it may be utilized by direct or modified use of the methods disclosed and/or referred to herein.

Acinetobacter calcoaceticus (DSMZ # 1139) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended A. calcoaceticus culture are made into BHI and are allowed to grow for aerobically for 48 hours at 37° C. at 250 rpm until saturated.

Bacillus subtilis is a gift from the Gill lab (University of Colorado at Boulder) and is obtained as an actively growing culture. Serial dilutions of the actively growing B. subtilis culture are made into Luria Broth (RPI Corp, Mt. Prospect, Ill., USA) and are allowed to grow for aerobically for 24 hours at 37° C. at 250 rpm until saturated.

Chlorobium limicola (DSMZ# 245) is obtained from the German Collection of Microorganisms and Cell Cultures (Braunschweig, Germany) as a vacuum dried culture. Cultures are then resuspended using Pfennig's Medium I and II (#28 and 29) as described per DSMZ instructions. C. limicola is grown at 25° C. under constant vortexing.

Citrobacter braakii (DSMZ # 30040) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. braakii culture are made into BHI and are allowed to grow for aerobically for 48 hours at 30° C. at 250 rpm until saturated.

Clostridium acetobutylicum (DSMZ # 792) is obtained from the German Collection of Microorganisms and Cell Cultures (Braunschweig, Germany) as a vacuum dried culture. Cultures are then resuspended in Clostridium acetobutylicum medium (#411) as described per DSMZ instructions. C. acteobutylicum is grown anaerobically at 37° C. at 250 rpm until saturated.

Clostridium aminobutyricum (DSMZ # 2634) is obtained from the German Collection of Microorganisms and Cell Cultures (Braunschweig, Germany) as a vacuum dried culture. Cultures are then resuspended in Clostridium aminobutyricum medium (#286) as described per DSMZ instructions. C. aminobutyricum is grown anaerobically at 37° C. at 250 rpm until saturated.

Clostridium kluyveri (DSMZ #555) is obtained from the German Collection of Microorganisms and Cell Cultures (Braunschweig, Germany) as an actively growing culture. Serial dilutions of C. kluyveri culture are made into Clostridium kluyveri medium (#286) as described per DSMZ instructions. C. kluyveri is grown anaerobically at 37° C. at 250 rpm until saturated.

Cupriavidus metallidurans (DMSZ # 2839) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. metallidurans culture are made into BHI and are allowed to grow for aerobically for 48 hours at 30° C. at 250 rpm until saturated.

Cupriavidus necator (DSMZ # 428) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. necator culture are made into BHI and are allowed to grow for aerobically for 48 hours at 30° C. at 250 rpm until saturated.

Desulfovibrio fructosovorans (DSMZ # 3604) is obtained from the German Collection of Microorganisms and Cell Cultures (Braunschweig, Germany) as a vacuum dried culture. Cultures are then resuspended in Desulfovibrio fructosovorans medium (#63) as described per DSMZ instructions. D. fructosovorans is grown anaerobically at 37° C. at 250 rpm until saturated.

Escherichia coli Crooks (DSMZ#1576) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended E. coli Crooks culture are made into BHI and are allowed to grow for aerobically for 48 hours at 37° C. at 250 rpm until saturated.

Escherichia coli t K12 is a gift from the Gill lab (University of Colorado at Boulder) and is obtained as an actively growing culture. Serial dilutions of the actively growing E. coli K12 culture are made into Luria Broth (RPI Corp, Mt. Prospect, Ill., USA) and are allowed to grow for aerobically for 24 hours at 37° C. at 250 rpm until saturated.

Halobacterium salinarum (DSMZ# 1576) is obtained from the German Collection of Microorganisms and Cell Cultures (Braunschweig, Germany) as a vacuum dried culture. Cultures are then resuspended in Halobacterium medium (#97) as described per DSMZ instructions. H. salinarum is grown erobically at 37° C. at 250 rpm until saturated.

Lactobacillus delbrueckii (#4335) is obtained from WYEAST USA (Odell, Oreg., USA) as an actively growing culture. Serial dilutions of the actively growing L. delbrueckii culture are made into Brain Heart Infusion (BHI) broth (RPI Corp, Mt. Prospect, Ill., USA) and are allowed to grow for aerobically for 24 hours at 30° C. at 250 rpm until saturated.

Metallosphaera sedula (DSMZ #5348) is obtained from the German Collection of Microorganisms and Cell Cultures (Braunschweig, Germany) as an actively growing culture. Serial dilutions of M. sedula culture are made into Metallosphaera medium (#485) as described per DSMZ instructions. M. sedula is grown aerobically at 65° C. at 250 rpm until saturated.

Propionibacterium freudenreichii subsp. shermanii (DSMZ# 4902) is obtained from the German Collection of Microorganisms and Cell Cultures (Braunschweig, Germany) as a vacuum dried culture. Cultures are then resuspended in PYG-medium (#104) as described per DSMZ instructions. P. freudenreichii subsp. shermanii is grown anaerobically at 30° C. at 250 rpm until saturated.

Pseudomonas putida is a gift from the Gill lab (University of Colorado at Boulder) and is obtained as an actively growing culture. Serial dilutions of the actively growing P. putida culture are made into Luria Broth (RPI Corp, Mt. Prospect, Ill., USA) and are allowed to grow for aerobically for 24 hours at 37° C. at 250 rpm until saturated.

Streptococcus mutans (DSMZ# 6178) is obtained from the German Collection of Microorganisms and Cell Cultures (Braunschweig, Germany) as a vacuum dried culture. Cultures are then resuspended in Luria Broth (RPI Corp, Mt. Prospect, Ill., USA). S. mutans is grown aerobically at 37° C. at 250 rpm until saturated.

Subsection II: Gel Preparation, DNA Separation, Extraction and Ligation Methods:

Molecular biology grade agarose (RPI Corp, Mt. Prospect, Ill., USA) is added to 1×TAE to make a 1% Agarose: TAE solution. To obtain 50×TAE add the following to 900 mL of distilled water: add the following to 900 ml distilled H₂O: 242 g Tris base (RPI Corp, Mt. Prospect, Ill., USA), 57.1 ml Glacial Acetic Acid (Sigma-Aldrich, St. Louis, Mo., USA) and 18.6 g EDTA (Fisher Scientific, Pittsburgh, Pa. USA) and adjust volume to 1 L with additional distilled water. To obtain 1×TAE, add 20 mL of 50×TAE to 980 mL of distilled water. The agarose-TAE solution is then heated until boiling occurred and the agarose is fully dissolved. The solution is allowed to cool to 50° C. before 10 mg/mL ethidium bromide (Acros Organics, Morris Plains, N.J., USA) is added at a concentration of 5 ul per 100 mL of 1% agarose solution. Once the ethidium bromide is added, the solution is briefly mixed and poured into a gel casting tray with the appropriate number of combs (Idea Scientific Co., Minneapolis, Minn., USA) per sample analysis. DNA samples are then mixed accordingly with 5×TAE loading buffer. 5×TAE loading buffer consists of 5×TAE (diluted from 50×TAE as described above), 20% glycerol (Acros Organics, Morris Plains, N.J., USA), 0.125% Bromophenol Blue (Alfa Aesar, Ward Hill, Mass., USA), and adjust volume to 50 mL with distilled water. Loaded gels are then run in gel rigs (Idea Scientific Co., Minneapolis, Minn., USA) filled with 1×TAE at a constant voltage of 125 volts for 25-30 minutes. At this point, the gels are removed from the gel boxes with voltage and visualized under a UV transilluminator (FOTODYNE Inc., Hartland, Wis., USA).

The DNA isolated through gel extraction is then extracted using the QIAquick Gel Extraction Microcentrifuge and Vacuum Protocol and associated materials and reagents (Qiagen, Valencia Calif. USA). Similar methods are known to those skilled in the art.

The thus-extracted DNA then may be ligated into pSMART (Lucigen Corp, Middleton, Wis., USA), StrataClone (Stratagene, La Jolla, Calif., USA) or pCR2.1-TOPO TA (Invitrogen Corp, Carlsbad, Calif., USA) according to manufacturer's instructions. These methods are described in the next subsection of Common Methods.

Ligation Methods:

For ligations into pSMART vectors:

Gel extracted DNA is blunted using PCRTerminator (Lucigen Corp, Middleton, Wis., USA) according to manufacturer's instructions. Then 500 ng of DNA is added to 2.5 ul 4× CloneSmart vector premix, 1 ul CloneSmart DNA ligase (Lucigen Corp, Middleton, Wis., USA) and distilled water is added for a total volume of 10 ul. The reaction is then allowed to sit at room temperature for 30 minutes and then heat inactivated at 70° C. for 15 minutes and then placed on ice. E. cloni 10 G Chemically Competent cells (Lucigen Corp, Middleton, Wis., USA) are thawed for 20 minutes on ice. 40 ul of chemically competent cells are placed into a microcentrifuge tube and 1 ul of heat inactivated CloneSmart Ligation is added to the tube. The whole reaction is stirred briefly with a pipette tip. The ligation and cells are incubated on ice for 30 minutes and then the cells are heat shocked for 45 seconds at 42° C. and then put back onto ice for 2 minutes. 960 ul of room temperature Recovery media (Lucigen Corp, Middleton, Wis., USA) and places into microcentrifuge tubes. Shake tubes at 250 rpm for 1 hour at 37° C. Plate 100 ul of transformed cells on Luria Broth plates (RPI Corp, Mt. Prospect, Ill., USA) plus appropriate antibiotics depending on the pSMART vector used. Incubate plates overnight at 37° C.

For Ligations into StrataClone:

Gel extracted DNA is blunted using PCRTerminator (Lucigen Corp, Middleton, Wis., USA) according to manufacturer's instructions. Then 2 ul of DNA is added to 3 ul StrataClone Blunt Cloning buffer and 1 ul StrataClone Blunt vector mix amp/kan (Stratagene, La Jolla, Calif., USA) for a total of 6 ul. Mix the reaction by gently pipeting up at down and incubate the reaction at room temperature for 30 minutes then place onto ice. Thaw a tube of StrataClone chemically competent cells (Stratagene, La Jolla, Calif., USA) on ice for 20 minutes. Add 1 ul of the cloning reaction to the tube of chemically competent cells and gently mix with a pipette tip and incubate on ice for 20 minutes. Heat shock the transformation at 42° C. for 45 seconds then put on ice for 2 minutes. Add 250 ul pre-warmed Luria Broth (RPI Corp, Mt. Prospect, Ill., USA) and shake at 250 rpm for 37° C. for 2 hour. Plate 100 ul of the transformation mixture onto Luria Broth plates (RPI Corp, Mt. Prospect, Ill., USA) plus appropriate antibiotics. Incubate plates overnight at 37° C.

For Ligations into pCR2.1-TOPO TA:

Add 1 ul TOPO vector, 1 ul Salt Solution (Invitrogen Corp, Carlsbad, Calif., USA) and 3 ul gel extracted DNA into a microcentrifuge tube. Allow the tube to incubate at room temperature for 30 minutes then place the reaction on ice. Thaw one tube of TOP10 chemically competent cells (Invitrogen Corp, Carlsbad, Calif., USA) per reaction. Add 1 ul of reaction mixture into the thawed TOP10 cells and mix gently by swirling the cells with a pipette tip and incubate on ice for 20 minutes. Heat shock the transformation at 42° C. for 45 seconds then put on ice for 2 minutes. Add 250 ul pre-warmed SOC media (Invitrogen Corp, Carlsbad, Calif., USA) and shake at 250 rpm for 37° C. for 1 hour. Plate 100 ul of the transformation mixture onto Luria Broth plates (RPI Corp, Mt. Prospect, Ill., USA) plus appropriate antibiotics. Incubate plates overnight at 37° C.

Subsection III. HPLC Analytical Method

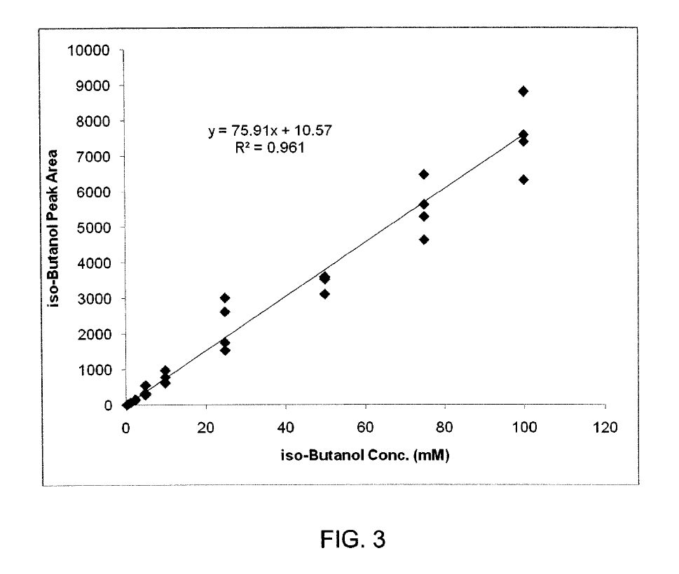

The Waters chromatography system (Milford, Mass.) consisted of the following: 600S Controller, 616 Pump, 717 Plus Autosampler, 410 Refractive Index (RI) Detector, and an in-line mobile phase Degasser. In addition, an Eppendorf external column heater is used and the data is collected using an SRI (Torrance, Calif.) analog-to-digital converter linked to a standard desk top computer. Data is analyzed using the SRI Peak Simple software. A Coregel Ion310 ion exclusion column (Transgenomic, Inc., San Jose, Calif.) is employed. The column resin is a sulfonated polystyrene divinyl benzene with a particle size of 8 μm and column dimensions are 150×6.5 mm. The mobile phase consists of sulfuric acid (Fisher Scientific, Pittsburgh, Pa. USA) diluted with deionized (18 MΩcm) water to a concentration of 0.02 N and vacuum filtered through a 0.2 μm nylon filter. The flow rate of the mobile phase is 0.6 mL/min. The RI detector is operated at a sensitivity of 128 and the column is heated to 60° C. The same equipment and method as described herein is used for the butanol and isobutanol analyses for relevant prophetic examples. Calibration curves using this HPLC method with butanol and isobutanol reagent grade standards (Sigma-Aldrich, St. Louis, Mo., USA) are provided in FIGS. 2 and 3.

Summary of Suppliers Section

This section is provided for a summary of suppliers, and may be amended to incorporate additional supplier information in subsequent filings. The names and city addresses of major suppliers are provided in the methods above. In addition, as to Qiagen products, the DNeasy® Blood and Tissue Kit, Cat. No. 69506, is used in the methods for genomic DNA preparation; the QIAprep® Spin (“mini prep”), Cat. No. 27106, is used for plasmid DNA purification, and the QIAquick® Gel Extraction Kit, Cat. No. 28706, is used for gel extractions as described above.

(End of Examples Section of the Specification)

The use of E. coli, although convenient for many reasons, is not meant to be limiting. One or more of the butanol and/or isobutanol biosynthetic pathways may be provided, by methods such as those described herein and generally known to those skilled in the art, to other microorganisms, such as bacterial and fungal species. Other candidate microorganisms that may be genetically engineered to comprise any such butanol and/or isobutanol biosynthetic pathway may include, but are not limited to: any gram negative microorganisms such s E. coli, or Pseudomononas sp.; any gram positive microorganism, for example Bacillus subtilis, Lactobaccilus sp. or Lactococcus sp. a yeast, for example Saccharomyces cerevisiae, Pichia pastoris or Pichia stipitis; and other groups or microbial species.

Microbial Hosts for Butanol and/or Isobutanol Bio-Production

Microbial hosts for butanol and/or isobutanol bio-production may be selected from bacteria, cyanobacteria, filamentous fungi and yeasts. The microbial host used for butanol and/or isobutanol bio-production is preferably tolerant to butanol and/or isobutanol so that the yield is not limited by butanol toxicity. Microbes that are metabolically active at high titer levels of butanol and/or isobutanol are not well known in the art.

The microbial host for butanol and/or isobutanol production should also utilize sugars including glucose at a high rate. Most microbes are capable of utilizing carbohydrates. However, certain environmental microbes cannot utilize carbohydrates to high efficiency, and therefore would not be suitable hosts without genetic manipulation.

The ability to genetically modify the host is essential for the production of any recombinant microorganism. The mode of gene transfer technology may be by electroporation, conjugation, transduction or natural transformation. A broad range of host conjugative plasmids and drug resistance markers are available. The cloning vectors are tailored to the host organisms based on the nature of antibiotic resistance markers that can function in that host.

The microbial host also has to be manipulated in order to inactivate competing pathways for carbon flow by deleting various genes. This requires the availability of either transposons to direct inactivation or chromosomal integration vectors. Additionally, the production host should be amenable to chemical mutagenesis so that mutations to improve intrinsic butanol and/or isobutanol tolerance may be obtained.

Based on the criteria described above, suitable microbial hosts for the production of butanol and/or isobutanol may include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae. However, in various aspects of the invention the microorganism is not Clostridium phytofermentans, and more particularly is not that species when a bio-production event provides more than 20 μM of a carbohydrate as a carbon source. In addition, it is contemplated that aspects of the present invention also may be practiced in one or more species of algae, such as single-cell or colonial types.

Bio-Production Media

Bio-production media, which is used in the present invention with recombinant microorganisms having a biosynthetic pathway for butanol and/or isobutanol, must contain suitable carbon substrates. Suitable substrates may include, but are not limited to, monosaccharides such as glucose and fructose, oligosaccharides such as lactose or sucrose, polysaccharides such as starch or cellulose or mixtures thereof and unpurified mixtures from renewable feed stocks such as cheese whey permeate, cornsteep liquor, sugar beet molasses, and barley malt. Additionally the carbon substrate may also be one-carbon substrates such as carbon dioxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sutter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, preferred carbon substrates are glucose, fructose, and sucrose, as well as mixtures of any of these sugars. Sucrose may be obtained from feed stocks such as sugar cane, sugar beets, cassaya, and sweet sorghum. Glucose and dextrose may be obtained through saccharification of starch based feed stocks including grains such as corn, wheat, rye, barley, and oats.

In addition, fermentable sugars may be obtained from cellulosic and lignocellulosic biomass through processes of pretreatment and saccharification, as described, for example, in US patent application US20070031918A1, which is herein incorporated by reference. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In addition to an appropriate carbon source, bio-production media must contain suitable minerals, salts, cofactors, buffers and other components, known to those skilled in the art, suitable for the growth of the cultures and promotion of the enzymatic pathway necessary for butanol and/or isobutanol production.

General Culture Conditions

Typically cells are grown at a temperature in the range of about 25° C. to about 40° C. in an appropriate medium. Suitable growth media in the present invention are common commercially prepared media such as Luria Bertani (LB) broth, M9 minimal media, Sabouraud Dextrose (SD) broth, Yeast medium (YM) broth or (Ymin) yeast synthetic minimal media. Other defined or synthetic growth media may also be used, and the appropriate medium for growth of the particular microorganism will be known by one skilled in the art of microbiology or bio-production science.

Suitable pH ranges for the bio-production are between pH 5.0 to pH 9.0, where pH 6.0 to pH 8.0 is preferred as the initial condition.

Bio-productions may be performed under aerobic, microaerobic or anaerobic conditions, with or without agitation.

The amount of butanol and/or isobutanol produced in the bio-production medium generally can be determined using a number of methods known in the art, for example, high performance liquid chromatography (HPLC) or gas chromatography (GC).

Bio-Production Reactors and Systems:

Any of the recombinant microorganisms as described and/or referred to above may be introduced into an industrial bio-production system where the microorganisms convert a carbon source into butanol and/or isobutanol in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to butanol and/or isobutanol. Industrial bio-production systems and their operation are well-known to those skilled in the arts of chemical engineering and bioprocess engineering. The following paragraphs provide an overview of the methods and aspects of industrial systems that may be used for the bio-production of butanol and/or isobutanol.

In various embodiments, any of a wide range of sugars, including, but not limited to sucrose, glucose, xylose, cellulose or hemixellulose, are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing one or more of the butanol and/or isobutanol biosynthetic pathway alternatives, and the a carbon source may be combined. The carbon source enters the cell and is cataboliized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3^{rd} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.). The appropriate intermediates are subsequently converted to butanol and/or isobutanol by one or more of the above-disclosed biosynthetic pathways.

Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the Fed-Batch system. Fed-Batch bio-production processes are also suitable in the present invention and comprise a typical batch system with the exception that the substrate is added in increments as the bio-production progresses. Fed-Batch systems are useful when catabolite repression is apt to inhibit the metabolism of the cells and where it is desirable to have limited amounts of substrate in the media. Measurement of the actual substrate concentration in Fed-Batch systems may be measured directly, such as by sample analysis at different times, or estimated on the basis of the changes of measurable factors such as pH, dissolved oxygen and the partial pressure of waste gases such as CO₂. Batch and Fed-Batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. 011 is, McGraw Hill, New York, 1986, herein incorporated by reference for general instruction on bio-production, which as used herein may be aerobic, microaerobic, or anaerobic, and with or without agitation.

Although the present invention may be performed in fed-batch mode it is contemplated that the method would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include: 1) Chemostat—where fresh media is fed to the vessel while simultaneously removing an equal rate of the vessel contents. The limitation of this approach is that cells are lost and high cell density generally is not achievable. In fact, typically one can obtain much higher cell density with a fed-batch process. 2) Perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the bio-production. Methods of modulating nutrients and growth factors for continuous bio-production processes as well as techniques for maximizing the rate of product formation are well known in the art of industrial microbiology and a variety of methods are detailed by Brock, supra.

It is contemplated that embodiments of the present invention may be practiced using either batch, fed-batch or continuous processes and that any known mode of bio-production would be suitable. Additionally, it is contemplated that cells may be immobilized on an inert scaffold as whole cell catalysts and subjected to suitable bio-production conditions for butanol and/or isobutanol production.

The following published resources are incorporated by reference herein for their respective teachings to indicate the level of skill in these relevant arts, and as needed to support a disclosure that teaches how to make and use methods of industrial bio-production of butanol and/or isobutanol from sugar sources, and also industrial systems that may be used to achieve such conversion with any of the recombinant microorganisms of the present invention (Biochemical Engineering Fundamentals, 2^{nd} Ed. J. E. Bailey and D. F. 011 is, McGraw Hill, New York, 1986, entire book for purposes indicated and Chapter 9, pages 533-657 in particular for biological reactor design; Unit Operations of Chemical Engineering, 5^{th} Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings).

At conclusion of a bio-production event the butanol and/or isobutanol, which may be obtained at least in a measurable quantity, is separated from the final bio-production solution (which may comprise solids in the liquid) by any of the separation means known in the art. As appropriate, when both butanol and isobutanol are present, they may be separated as is feasible given the economics of this separation in view of the downstream uses of these products.

The above discloses and teaches methods, compositions, and systems that provide for various approaches to microbial bio-production of butanol and/or isobutanol. It is appreciated that as the titer of butanol and/or isobutanol gets higher it exerts a growth-inhibiting and/or toxic effect on microorganisms in the respective culture or industrial system. Any of a number of strategies and methods may be employed to determine the cause(s) and mechanism(s) of such undesired effect(s), and/or to identify genes and/or nucleic acid sequences, that when expressed, result in greater tolerance to butanol and/or isobutanol. Techniques that are contemplated to obtain higher-tolerant microorganism under environmental pressure, such as in the presence of butanol and/or isobutanol, include those described in WO/2007/130560. For example an enrichment culture is grown at a temperature of about 25° C. to about 60° C. for a time sufficient for the members of the microbial culture in a sample (such as obtained from a location historically exposed to butanol, isobutanol, or a similar alcohol) to exhibit growth, typically about 12 hours to about 24 hours. The culture may be grown under anaerobic, microaerobic, or aerobic conditions, with or without agitation. The growing enrichment culture is then contacted with butanol and/or isobutanol. This contacting may be done by diluting the enrichment culture with a fresh growth medium that contains butanol. The microbial culture that was contacted with butanol is then separated to isolate individual strains. Contacting a microbial culture with butanol and/or isobutanol together with a mutagen, such as nitrosoguanidine (NG), such as in the center of a Petri dish, which creates a desired gradient by progressive diffusion of the mutagenesis agent, may also be practiced to obtain a microorganism comprising a certain level of tolerance to butanol and/or isobutanol (See, e.g., U.S. Pat. No. 4,757,010).

However, various genomics and other more sophisticated strategies and methods may also be used to identify and/or improve tolerance mechanisms. Among the genomics approaches to identifying tolerance-related genes and/or nucleic acid sequences is a method described in U.S. Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” (hereinafter, the “Gill et al. Technique”), which are incorporated herein by reference in their entirety for the teaching of the technique.

To obtain genetic information used for analysis that results in identification and utilization of tolerance-improving genetic modification(s), initially butanol or isobutanol-related fitness data is obtained by evaluation of fitness of clones from a genomic-library population using the SCALES technique. This technique is cited in the Background section, above, and is described in greater detail in paragraphs below. Accordingly, the following paragraphs describe a technique that may be employed to acquire genetic data that is analyzed, the analysis resulting in making the discoveries that allow for identification of genetic elements relevant to butanol and/or isobutanol tolerance. That is, the purpose is to identify which genes or other nucleic acid sequences are related to increased fitness for tolerance of butanol or isobutanol.

More particularly, to obtain data potentially useful to identify genetic elements relevant to increased butanol or isobutanol tolerance, an initial population of five representative E. coli K12 genomic libraries is produced by methods known to those skilled in the art. The five libraries respectively comprise 500, 1000, 2000, 4000, 8000 base pair (“bp”) inserts of E. coli K12 genetic material. Each of these libraries, essentially comprising the entire E. coli K12 genome, is respectively transformed into MACH1-TR and cultured to about mid-exponential phase. The culture conditions are maintained aerobic and batch transfer times are constant. Although not meant to be limiting as to alternative approaches, selection in the presence of butanol or isobutanol is carried out over 4-10 serial transfer batches with an increasing or a decreasing gradient of butanol or isobutanol over 60 hours. Samples are taken during and at the culmination of each batch in the selection, and are subjected to microarray analysis that identifies signal strengths. The individual methods for preparing libraries, transformation of cell cultures, and other methods used for the SCALES technique prior to array and data analyses are well-known in the art, such as supported by methods taught in Sambrook and Russell, Molecular Cloning: A Laboratory Manual, Third Edition 2001 (volumes 1-3), Cold Spring Harbor Laboratory Press, Cold Spring Harbor, N.Y. Aspects of individual methods also are discussed in greater detail in the SCALES technique references, U.S. Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” (hereinafter, the “SCALES Technique”), which are incorporated herein by reference for the teaching such details of this technique.

Microarray technology also is well-known in the art (see, e.g. www.affymetrix.com). To obtain data of which clones are more prevalent at different exposure periods to butanol or isobutanol, Affymetrix E. Coli Antisense Gene Chip arrays (Affymetrix, Santa Clara, Calif.) are handled and scanned according to the E. Coli expression protocol from Affymetrix producing affymetrix .cel files. A strong microarray signal after a given exposure to butanol or isobutanol could indicate that the genetic sequence introduced by the plasmid to this clone correlates with butanol or isobutanol tolerance. The microarray data is analyzed with software suited for the SCALES technique in order to decompose the microarray signals into corresponding library clones and calculate relative enrichment of specific regions over time. In this way, genome-wide fitness (In(Xᵢ/Xᵢ₀)) is measured based on region specific enrichment patterns for the selection in the presence of the industrially relevant organic acid, butanol or isobutanol. For example, in some evaluations probe level signals are extracted from the Affymetrix .cel files using the Expression Exporter software (Affymetrix). For each array, in order to subtract background signal as well as any signal from genomic DNA contamination, the largest signal from any non-loaded control probe is subtracted from all probes. Next, outlier probes are identified and are removed using a Hampel or other suitable identifier, with probes signals averaged over a 250 bp range to calculate median values. Average signals of positive control probes are fit to a logarithmic function of moles. This is used to calculate the moles due to each signal in the sample. These signals are then mapped to genomic position giving a signal as a function of position. Data is padded by filling genomic positions between probes with a line connecting closest probe pairs. The resulting signal is subjected to a continuous wavelet transform to perform the multiresolution analysis. Every 10 base pairs is given a signal. This signal is subjected to a discrete wavelet transform using a Debauchies mother wavelet and WaveLab v. 8.02 Software (Rice University), or other suitable transformation approach. The signal is reconstructed after deletion of scales smaller than 500 bp. The resulting denoised signal is subjected to a multiresolution analysis using the same or similar software.

This approach provides data for the analysis that leads identification of genetic elements whose increased expression (based on increased copy number via a respective plasmid) positively correlates with increased tolerance to butanol or isobutanol. The data may be combined with data from other approaches for determining tolerance to butanol and/or isobutanol to obtain valuable information and also to develop recombinant microorganisms that comprise genetic modification(s) providing elevated butanol tolerance and/or isobutanol tolerance compared to a control microorganism lacking such genetic modification(s).

In a prophetic example, practicing the SCALES method for butanol and/or isobutanol tolerance, optionally in combination with other approaches to obtaining or determining tolerance features in a microorganism, provides data that is used to identify specific genetic elements, such as genes or other nucleic acid sequences, and one or more genetic modifications are made to a microorganism that introduce one or more copies nucleic acid sequences related to such genes or other nucleic acid sequences. After such genetic modification(s) the recombinant microorganism exhibits increased tolerance to butanol and/or isobutanol.

Accordingly, the present invention may include a recombinant microorganism, and a method of butanol and/or isobutanol production, comprising any of the butanol and/or isobutanol biosynthesis pathway alternatives described above, particularly those alternatives that include an enzyme that effectively ‘bridges’ pathways A and B between butyryl-CoA and isobutyrl-CoA (e.g., isobutyryl-CoA mutase), that further comprise one or more genetic modifications providing increased tolerance to butanol and/or isobutanol. Standard selection methods may be used to identify a more tolerant organism (into which nucleic acid sequences for production pathways may be introduced), and/or analysis of data obtained from the referenced Gill et al. technique, or from other known techniques, may identify genetic elements related to increased tolerance. These genetic elements may be introduced into a microorganism, along with genetic elements to provide and/or improve one or more of the butanol/isobutanol production pathway alternatives.

Thus, a recombinant microorganism according to the present invention may comprise any of the butanol and/or isobutanol production pathway alternatives described and/or taught herein, in various embodiments including the ‘bridge’, and genetic modifications directed to increased tolerance to butanol and/or isobutanol, to provide a recombinant microorganism that both produces and has increased tolerance to butanol and/or isobutanol. Such recombinant microorganism may demonstrate increased productivity and yield of butanol and/or isobutanol (compared with a non-modified control microorganism). Such ‘doubly-modified’ recombinant microorganism may be appreciated to have high commercial value for use in industrial systems that are designed to biosynthesize butanol and/or isobutanol in a cost-effective manner. Genetic modifications directed to reduce or eliminate bio-production of undesired intermediates and/or products, and/or mutant strains such as exemplified above by NZN111 and JW1375, may also be used in combination with genetic modifications directed to production, and to tolerance, of butanol and/or isobutanol.

At a relatively basic level, suitable host strains with a tolerance for butanol and/or isobutanol may be identified by screening based on the intrinsic tolerance of the strain. The intrinsic tolerance of microbes to butanol and/or isobutanol may be measured by determining the (MIC) or minimum inhibitory concentration of butanol and/or isobutanol that is responsible for complete inhibition of growth in a given environment and media. The MIC values may be determined using methods known in the art. In addition several other methods of determining microbial tolerance may be used, not limited to but including, minimum bacteriocidal concentration (MBC), the minimum concentration needed to completely kill all cells in a microbial culture in a given environment and media, or the 1050 or the concentration of butanol and/or isobutanol that is responsible for 50% inhibition of the growth rate (1050) when grown in a defined media and environment. The MIC, MBC and 1050 values may be determined using methods known in the art. For example, the microbes of interest may be grown in the presence of various amounts of butanol and/or isobutanol and the growth rate monitored by measuring the optical density at 600 nanometers. The doubling time may be calculated from the logarithmic part of the growth curve and used as a measure of the growth rate.

In summary, any of the solutions obtained that provide for greater tolerance to butanol and/or isobutanol may be applied to and combined with any of the above-disclosed biosynthesis alternative approaches and/or genetic modifications that reduce or eliminate production of undesired metabolic products.

Accordingly, it is within the presently conceived scope of the invention, at least for some embodiments, to genetically modify a microorganism of interest to comprise both 1) one or more introduced genetic elements (i.e., heterologous nucleic acid sequences) providing enzymatic function to complete one of the butanol and/or isobutanol biosynthetic pathways described herein (and such as are claimed herein), and 2) one or more introduced genetic elements (i.e., heterologous nucleic acid sequences) providing enzymatic function(s) directed to increasing the microorganism's tolerance to butanol and/or isobutanol, and optionally also 3) one or more genetic modification(s) directed to reduce or eliminate production of metabolic products other than butanol and/or isobutanol. Improvement of tolerance to butanol and/or isobutanol by a recombinant butanol and/or isobutanol-synthesizing microorganism generally is considered of value in order to achieve more cost-effective industrial systems for butanol and/or isobutanol biosynthesis. This is related at least in part to higher downstream separation costs when butanol and/or isobutanol final titers are relatively low at the end of an industrial system biosynthetic process.

Accordingly, based on the above discussion and teachings, the scope of the present invention includes producing butanol and/or isobutanol by any combination of the above pathways and alternatives and their variations. Further, the various embodiments of the present invention may include further genetic modifications, such as by use and modification of a known mutant microorganism (such as NZN111), or genetic modification such as by deletion, addition, substitution, etc., as is known to those skilled in the art, so that the production of an undesired competing metabolic product, which may be referred to herein as “other metabolic product,” is reduced or eliminated. Further, embodiments comprising one of the butanol and/or isobutanol biosynthesis pathway alternatives, particularly comprising the ‘bridge,’ may include a tolerance-improving mechanism, whether the latter is implemented by a genetic modification and/or a modification to the culture system, wherein that mechanism improves microorganism tolerance to butanol and/or isobutanol.

The scope of the present invention is not meant to be limited to the exact sequences provided herein. It is appreciated that a range of modifications to nucleic acid and to amino acid sequences (e.g., polypeptides and enzymes comprising enzymatic activity, such as for the genes and enzyme functions described above), may be made and still provide a desired functionality. The following discussion is provided to more clearly define ranges of variation that may be practiced and still remain within the scope of the present invention.

It is recognized in the art that some amino acid sequences of the present invention can be varied without significant effect of the structure or function of the proteins disclosed herein. Variants included can constitute deletions, insertions, inversions, repeats, and type substitutions so long as the indicated enzyme activity is not significantly affected. Guidance concerning which amino acid changes are likely to be phenotypically silent can be found in Bowie, J. U., et Al., “Deciphering the Message in Protein Sequences Tolerance to Amino Acid Substitutions,” Science 247:1306-1310 (1990).

In various embodiments polypeptides obtained by the expression of the polynucleotide molecules of the present invention may have at least approximately 80%, 90%, 95%, 96%, 97%, 98%, 99% or 100% identity to one or more amino acid sequences encoded by the genes and/or nucleic acid sequences described herein for the butanol and/or isobutanol biosynthesis pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

For example, in a specific embodiment the identity between a reference sequence (query sequence, a sequence of the present invention) and a subject sequence, also referred to as a global sequence alignment, may be determined using the FASTDB computer program based on the algorithm of Brutlag et al. (Comp. App. Biosci. 6:237-245 (1990)). Preferred parameters used in a FASTDB amino acid alignment are: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

Accordingly, it is within the scope of the invention to provide and use a genetically modified microorganism that comprises a polypeptide encoded by a heterologous nucleic acid sequence has at least a 90% homology, or at least a 95% homology, with apolypeptide encoded by any nucleic acid sequence disclosed herein, such as those described above, noted in FIG. 1, and including those for which sequence listings are provided herewith.

The above descriptions and methods for sequence homology are intended to be exemplary and it is recognized that this concept is well-understood in the art. Further, it is appreciated that nucleic acid sequences may be varied and still provide a functional enzyme, and such variations are within the scope of the present invention. Nucleic acid sequences that encode polypeptides that provide the indicated functions for butanol and/or isobutanol increased tolerance or production are considered within the scope of the present invention. These may be further defined by the stringency of hybridization, described below, but this is not meant to be limiting when a function of an encoded polypeptide matches a specified butanol and/or isobutanol tolerance-related or biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook and Russell and Anderson “Nucleic Acid Hybridization” 1^{st} Ed., BIOS Scientific Publishers Limited (1999), which are hereby incorporated by reference for hybridization protocols. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Embodiments of the present invention may result from introduction of an expression vector into a host microorganism, wherein the expression vector contains a nucleic acid sequence coding for an enzyme that is, or is not, normally found in a host microorganism. With reference to the host microorganism's genome, then, the nucleic acid sequence that codes for the enzyme is heterologous.

Also, and more generally, in accordance with examples and embodiments herein, there may be employed conventional molecular biology, cellular biology, microbiology, and recombinant DNA techniques within the skill of the art. Such techniques are explained fully in the literature. (See, e.g., Sambrook and Russell, Molecular Cloning: A Laboratory Manual, Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Further, all patents, patent applications, patent publications, and other publications referenced herein (collectively, “published resource(s)”) are hereby incorporated by reference in this application. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention.

While various embodiments of the present invention have been shown and described herein, it will be obvious that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated herein in a list, table, or other grouping, unless clearly stated otherwise, it is intended that each such grouping provides the basis for and serves to identify various subset embodiments, the subset embodiments in their broadest scope comprising every subset of such grouping by exclusion of one or more members of the respective stated grouping. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

1-38. (canceled)

39. A recombinant microorganism comprising at least one genetic modification to provide nucleic acids that encode polypeptides that catalyze one or more conversions from acetyl-CoA or pyruvate to isobutanol, wherein conversion of isobutyryl-CoA to isobutanol requires at least one of an aldehyde dehydrogenase and an aldehyde dehydrogenase.

40. The recombinant microorganism of claim 39 that comprises an isobutyryl-CoA mutase polypeptide.

41. The recombinant microorganism of claim 39 that comprises an aldehyde dehydrogenase derived from Rattus norvegicus.

42. The recombinant microorganism of claim 41 that comprises a 3-hydroxyisobutyrate hydrolase.

43. The recombinant microorganism of claim 39 that comprises an aldehyde dehydrogenase of Giardia lamblia.

44. A method for the biosynthesis of isobutanol comprising providing in a bioreactor vessel a recombinant microorganism of claim 39, a carbon source, and a media, and conducting a bio-production event to obtain isobutanol.

45. The method of claim 44 wherein the carbon source is selected from the group consisting of monosaccharides, oligosaccharides, and polysaccharides.

46. The method of claim 44 wherein the carbon source is selected from the group consisting of glucose, sucrose, and fructose.

47. The method of claim 44 wherein the media is a minimal media.

48. The method of claim 44 wherein the recombinant microorganism is selected from a bacterium and a yeast.

49. The method of claim 44 wherein the recombinant microorganism is selected from members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces.

50. An industrial-scale microbial bioreactor system comprising:
a. a bioreactor vessel;
b. a carbon source;
c. a recombinant microorganism of claim 39; and
d. a media.

51. The industrial-scale microbial bioreactor system of claim 50, wherein the media is minimal media.

52. The industrial-scale microbial bioreactor system of claim 50, wherein the recombinant microorganism comprises an isobutyryl-CoA mutase polypeptide and may produce butanol in addition to isobutanol.

53. A recombinant microorganism comprising at least one genetic modification to provide nucleic acids that encode polypeptides that catalyze one or more conversions from acetyl-CoA or pyruvate to butanol.

54. The recombinant microorganism of claim 53 that comprises an isobutyryl-CoA mutase polypeptide.

55. The recombinant microorganism of claim 53 that additionally is adapted to convert isobutyryl-CoA to isobutanol using at least one of an aldehyde dehydrogenase and an aldehyde dehydrogenase.

56. The recombinant microorganism of claim 39 additionally comprising a genetic modification of a nucleic acid encoding at least one enzyme, effective to decrease or eliminate bio-production of a metabolic product other than isobutanol.

57. The recombinant microorganism of claim 53 additionally comprising a genetic modification of a nucleic acid encoding at least one enzyme, effective to decrease or eliminate bio-production of a metabolic product other than butanol.
